(19) United States
(12) Patent Application Publication
(10) Pub. No.: US 2026/0306428 A1
(43) Pub. Date: Oct. 8, 2026

(54) METHODS OF PREVENTING POST-TRAUMATIC OSTEOARTHRITIS USING METFORMIN

(71) Applicant: The Trustees of Indiana University, Bloomington, IN (US)

(72) Inventors: Diane Renee Wagner, Indianapolis, IN (US); Todd McKinley, Indianapolis, IN (US); Roman Michael Natoli, Indianapolis, IN (US); Uma Sankar-Davis, Bloomington, IN (US); Sonali J. Karnik, Indianapolis, IN (US)

(21) Appl. No.: 19/483,255

(22) PCT Filed: May 10, 2024
(86) PCT No.: PCT/US2024/028754

§ 371 Date: Nov. 11, 2025

Related U.S. Application Data
(60) Provisional application No. 63/465,651, filed on May 11, 2023.

(51) Int. Cl. A61K 31/155; A61K 9/00; A61K 9/06; A61K 31/17; A61K 31/4965; A61K 31/513; A61K 31/522; A61K 31/70; A61K 31/7042; A61K 31/7048; A61K 47/36; A61P 19/02

(52) U.S. Cl. CPC A61K 31/155; A61K 9/0019; A61K 9/06; A61K 31/17; A61K 31/4965; A61K 31/513; A61K 31/522; A61K 31/70; A61K 31/7042; A61K 31/7048; A61K 47/36; A61P 19/02

(57) ABSTRACT

Disclosed herein are methods of using metformin or a pharmaceutically acceptable salt thereof, such as intraarticular administration of metformin, to treat injured joints. Also disclosed are methods of using metformin to prevent the progression of osteoarthritis, such as osteoarthritis resulting from an injury, or post-traumatic osteoarthritis. Further disclosed are dosing regimens and compositions of metformin for the treatment and prevention of post-traumatic osteoarthritis.

### CROSS-REFERENCE TO RELATED APPLICATION

The present application claims priority to U.S. Provisional Application No. 63/465,651, filed on May 11, 2023, and entitled “METHODS OF PREVENTING POST-TRAUMATIC OSTEOARTHRITIS USING METFORMIN,” the entire disclosure of which is expressly incorporated by reference herein.

### STATEMENT OF GOVERNMENTAL RIGHTS

This invention was made with government support under AR069657 and AR080255 awarded by the National Institutes of Health (NIH). The Government has certain rights in the invention.

### FIELD OF THE INVENTION

This disclosure generally relates to methods of administering metformin to treat injured joints and prevent the progression of cartilage damage to osteoarthritis, such as post-traumatic osteoarthritis. This disclosure further relates to metformin dosing regimens and compositions for the same.

### BACKGROUND

High-energy trauma to an articular joint dramatically increases the risk of osteoarthritis (OA). The consequences of joint trauma leading to post-traumatic osteoarthritis (PTOA) are devastating, including joint pain, joint stiffness, and loss of mobility. PTOA is implicated in general injuries, e.g., sports injuries, and is the number one disabling condition among military personnel. For context, PTOA accounts for 92% of all arthritis in active-duty soldiers, and about 28% of combat-wounded soldiers experience some degree of disability due to PTOA. Additionally, in the general U.S. population, joint trauma is estimated to be responsible for over 6 million cases of osteoarthritis (OA), at a staggering cost of $15 billion annually. Cases involving joint trauma are most common in younger patients, and current treatments have poor long-term outcomes.

Unlike many degenerative conditions in which the time of onset is unknown, the majority of PTOA results from a single event. High-energy trauma to the joint results in a variety of clinical conditions such as joint fracture, ruptured ligaments, and damaged meniscus in the knee. However, in all cases, the cartilage tissue experiences a mechanical joint overload, which causes high levels of impact-associated acute mechanical damage (AMD) to the cartilage tissue. Additionally, intraarticular fracture and damage to ligaments and/or menisci disrupts normal stress transfer during loading between the articular surfaces, thereby altering the chronic residual mechanical environment (RME).

Although both AMD and RME contribute to PTOA, current treatments nearly exclusively focus on addressing the RME, e.g., by attempting to surgically restore the native articular anatomy. Unfortunately, this narrow approach fails to address the polyetiology of PTOA. Consequently, the incidence of PTOA remains high, and the rate of fair to poor clinical outcomes has not effectively changed in decades. Treatments and therapeutic regimens that effectively address the multiple physiological consequences of acute joint injury, such as additionally reducing inflammation, preserving chondrocyte function, and mitigating AMD post-impact, are necessary to prevent the serious burdens posed by PTOA.

In experimental studies, the antihyperglycemic agent metformin has shown efficacy in preventing the progression of OA, including in traumatic models of OA. However, further insight into the underlying mechanisms of metformin's efficacy and safety in the context of treating injured chondrocytes is necessary to inform effective dosing strategies. In one aspect, the therapeutic ceiling of metformin across various routes of administration serves to undermine the effectiveness of current strategies, necessitating improved methods of using metformin to mitigate and prevent the progression of cartilage injury to OA, specifically PTOA. Aspects of the invention disclosed herein address these needs.

### INCORPORATION BY REFERENCE

Each patent, publication, and non-patent literature cited in the application is hereby incorporated by reference in its entirety as if each was incorporated by reference individually, and as if each is fully set forth herein. However, where such reference is made, and whether to patents, publications, non-patent literature, or other sources of information, it is for the general purpose of providing context for discussing features of the invention. Accordingly, unless specifically stated otherwise, the reference is not to be construed as an admission that the document or underlying information, in any jurisdiction, is prior art, or forms part of the common general knowledge in the art.

### SUMMARY OF THE INVENTION

A first aspect of the invention is a method of treating an injured joint in a mammal, which includes administering an injectable composition which includes metformin or a pharmaceutically acceptable salt thereof, such as metformin HCl, to the injured joint via intraarticular injection.

A second aspect of the invention is a method of treating an injured joint in a mammal, which includes administering an injectable composition including metformin or a pharmaceutically acceptable salt thereof, such as metformin HCl, to the injured joint via intraarticular injection in combination with systemic administration of metformin.

A third aspect of the invention is an injectable composition which includes metformin or a pharmaceutically acceptable salt thereof, such as metformin hydrochloride, and where the composition is suitable for intraarticular injection.

A first embodiment is a method of treating an injured joint in a mammal, which includes administering an injectable composition which includes a pharmaceutically acceptable salt of metformin at a concentration of 25 mM to 150 mM to the injured joint via intraarticular injection.

A second embodiment is a method of treating an injured joint in a mammal, which includes administering an injectable composition containing metformin hydrochloride to the injured joint via intraarticular injection.

A third embodiment is a method of treating an injured joint in a mammal, which includes administering an injectable composition which contains metformin hydrochloride at a concentration of about 50 mM to 150 mM, inclusive, to the injured joint via intraarticular injection.

A fourth embodiment is a method of treating an injured joint in a mammal, which includes administering an injectable composition containing metformin hydrochloride at a concentration of about 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, 100 mM, 105 mM, 110 mM, 115 mM, 120 mM, or 125 mM, to the injured joint via intraarticular injection.

A fifth embodiment is a method of treating an injured joint in a mammal, which includes administering 2 to 3 mL of the injectable composition to injured joint via the intraarticular route.

A sixth embodiment is a method of treating an injured joint in a mammal where the injectable composition is administered via intraarticular administration to the injured joint within 1 week or within 2 weeks of the injury.

A seventh embodiment is a method of treating an injured joint in a mammal, which includes administering the injectable composition by intraarticular administration to the injured joint within 1 hour, within 12 hours, or within 24 hours from occurrence of the injury.

An eighth embodiment is a method of treating an injured joint in a mammal, which includes administering the injectable composition via intraarticular injection to an injured wrist joint, an injured shoulder joint, an injured knee joint, an injured hip joint, an injured ankle joint, or an injured elbow joint in the mammal.

A ninth embodiment is a method of treating an injured joint in a mammal, which includes administering the injectable composition via intraarticular injection, where the injectable composition further comprises a hydrogel and a pharmaceutically acceptable excipient.

A tenth embodiment is a method of treating an injured joint in a mammal, where the hydrogel includes hyaluronic acid or a salt thereof, and where the salt is sodium hyaluronate or potassium hyaluronate.

An eleventh embodiment is a method of treating an injured joint in a mammal, where the injectable composition consists essentially of metformin hydrochloride and hyaluronic acid, metformin hydrochloride and sodium hyaluronate, or metformin hydrochloride and potassium hyaluronate.

A twelfth embodiment a method of treating an injured joint in a mammal, which further includes systemically administering an amount of a systemic formulation of metformin to the mammal, where the systemic formulation of metformin includes a pharmaceutically acceptable salt of metformin selected from the group consisting of metformin benzoate, metformin ethanesulfonate, metformin (2:1) fumarate, metformin glycinate, metformin glycolate, metformin hydrobromide, metformin hydrochloride, metformin maleate, metformin phosphate, metformin salicylate, metformin (2:1) succinate, and metformin sulfate.

A thirteenth embodiment is a method of treating an injured joint in a mammal, where the systemic formulation of metformin further comprises any one or more of alogliptin, canagliflozin, dapagliflozin, empagliflozin, ertugliflozin, glipizide, glyburide, linagliptin, and pharmaceutically acceptable salts thereof.

A fourteenth embodiment is a method of treating an injured joint in a mammal, where the amount of the pharmaceutically acceptable salt of metformin in the systemic formulation is up to about 3000 mg.

A fifteenth embodiment is a method of treating an injured joint in a mammal, where the amount of the pharmaceutically acceptable salt of metformin in the systemic formulation is about 100 mg to 2000 mg.

A sixteenth embodiment is a method of treating an injured joint in a mammal, where the amount of the pharmaceutically acceptable salt of metformin in the systemic formulation is about 1000 mg.

A seventeenth embodiment is a method of treating an injured joint in a mammal, where systemic administration includes oral administration, intramuscular (IM) administration, intravenous (IV) administration, or any combination thereof.

An eighteenth embodiment is a method of treating an injured joint in a mammal, where systemic administration includes only oral administration of metformin, not intramuscular (IM) administration, intravenous (IV) administration, nor any combination thereof.

A nineteenth embodiment is a method of treating an injured joint in a mammal, where the injured joint is a wrist joint, a shoulder joint, knee joint, a hip joint, an ankle joint, or an elbow joint.

A twentieth embodiment is a method of treating an injured joint in a mammal, where the joint injury comprises a fracture, cartilage damage, acute ligament sprain, chronic ligamentous instability, tendonitis, or a combination thereof.

A twenty-first embodiment is a method of treating an injured joint in a mammal, which additionally includes administering the injectable composition of metformin and/or the systemic formulation of metformin to a joint in a mammal prior to joint injury, wherein the joint is any one or more of a wrist joint, a shoulder joint, a knee joint, a hip joint, an ankle joint, and an elbow joint.

A twenty-second embodiment is a method of treating an injured joint in a mammal, where at least one symptom of post-traumatic osteoarthritis is prevented or reduced in the injured joint following treatment with metformin, wherein the at least one symptom is cartilage degeneration, joint pain, joint stiffness, inflammation, or a combination thereof.

A twenty-third embodiment is a method of treating an injured joint in a mammal, where prevention or reduction of the at least one symptom of post-traumatic osteoarthritis is determined by the presence of biomarkers, gait analysis, imaging analysis, histological analysis, a self-reported scale, or a combination thereof.

A twenty-fourth embodiment is a method of treating an injured joint in a mammal, where the mammal is a human.

A twenty-fifth embodiment is an injectable composition consisting essentially of hyaluronic acid or a salt thereof, wherein the salt is sodium hyaluronate or potassium hyaluronate, and at least 50 mM of metformin or a pharmaceutically acceptable salt thereof, wherein the pharmaceutically acceptable salt of metformin is metformin hydrochloride, and the composition is suitable for intraarticular injection.

A twenty-sixth embodiment is an injectable composition which includes 50 mM to 150 mM metformin hydrochloride, where the range is inclusive.

A twenty-seventh embodiment is an injectable composition, where intraarticular injection of the composition prevents at least one symptom of post-traumatic osteoarthritis in a mammal with an injured joint, wherein the at least one symptom is cartilage degeneration, joint pain, joint stiffness inflammation, or a combination thereof.

A twenty-eighth embodiment is use of the disclosed injectable composition, such as the injectable compositions in any of the preceding embodiments, for use in the treatment or the prevention of post-traumatic osteoarthritis.

A twenty-ninth embodiment is use of the disclosed injectable composition, such as the injectable compositions in any of the preceding embodiments, for the manufacture of a medicament for treating or preventing post-traumatic osteoarthritis.

### BRIEF DESCRIPTION OF THE FIGURES

The accompanying drawings are included to provide a further understanding of the disclosure and are incorporated in and constitute a part of this specification, illustrate embodiments, and together with the description serve to explain the principles of the disclosure.

FIG. 1A. Representative confocal images of untreated non-impacted cartilage explants, untreated sublethal impacted cartilage explants, and sublethal impacted cartilage explants treated with metformin (1 mM, 5 mM, and 10 mM MET) stained to detect apoptosis via caspase 3/7signaling.

FIG. 1B. Representative confocal images of untreated non-impacted cartilage explants, untreated sublethal impacted cartilage explants, and sublethal impacted cartilage explants treated with metformin (1 mM, 5 mM, and 10 mM MET) stained for reactive oxygen species (ROS).

FIG. 1C. Bar graph shows the corresponding quantification in fluorescence units of caspase 3/7signaling (top) and ROS (bottom) (* indicates p<0.05, a significant difference from non-impacted control).

FIG. 2A. Representative confocal images of impacted bovine cartilage show live cells stained green with calcein-AM and dead cells stained red with ethidium homodimer-1 in untreated impacted chondrocytes and impacted chondrocytes exposed to metformin (10 mM and 50 mM).

FIG. 2B. Bar graph shows the corresponding quantification of cell viability (*** and *** indicate a significant difference from the untreated impacted specimen, p<0.001 and p<0.0001, respectively).

FIG. 3A. Representative confocal images show TMRM (red indicating functional mitochondria) and MitoTracker Green (green indicating all mitochondria) staining of mitochondria in untreated non-impacted bovine cartilage and impacted bovine cartilage, untreated and exposed to metformin (10 mM and 50 mM).

FIG. 3B. Bar graph shows quantification of the corresponding red: green (R:G) fluorescent ratio in cartilage (* and ** indicate significant difference from non-impacted controls, p<0.05 and p<0.01, respectively).

FIG. 4A. Bar graph shows quantification of chondrocyte viability from live/dead staining of untreated cartilage explants and impacted cartilage explants treated with AMPK activator EX229 (1 μM and 10 μM) (* indicates p<0.05, a significant difference from the untreated impacted control).

FIG. 4B. Bar graph depicts quantification of chondrocyte viability from live/dead staining of untreated non-impacted and impacted cartilage explants, and impacted cartilage explants treated with SIRT1 activator SRT1720 (1 μM and 5 μM) (**, ***, and **** indicate a significant difference from the non-impacted and untreated control, p<0.01, p<0.001, and p<0.0001, respectively).

FIG. 5. Bar graph shows the red: green (R:G) intensity ratio of mitochondria stained with TMRM (red indicating functional mitochondria) and MitoTracker Green (all mitochondria) in non-impacted and impacted bovine cartilage cultured with metformin (125 mM).

FIG. 6. Representative images depict safranin-O staining of non-impacted (left) and impacted (right) medial condyles from rabbit knees.

FIG. 7. Representative images depict safranin-O staining of impacted rabbit knees treated with intraarticular injection of hydrogel alone (top) or intraarticular injection of hydrogel containing 50 mM metformin (bottom).

### DETAILED DESCRIPTION

Impact and injury to a joint can result in chondrocyte dysfunction and death, cartilage matrix disruption, and tissue loss, which are characteristic elements of osteoarthritis, specifically post-traumatic osteoarthritis (PTOA). In one example, trauma is pivotal to the development of PTOA in the knee, with individuals who sustain such an injury having a more than five-fold increased risk of developing the disease. For context, individuals who are overweight or obese have a 1.7-fold relative risk of developing osteoarthritis (OA). See Gelber, Ann. Intern. Med. 2000; 133, 321-328 and Gelber, Am. J. Med. 1999; 107, 542-548.

The chondrocyte response to injury and AMD involves a complex interplay of mechanical, cellular, and biochemical processes that can cause progressive cartilage loss (Carbone & Rodeo, J. Orthop. Res., 2017; 35:397-405). Chondrocytes play a vital role in cartilage homeostasis, and mitochondrial dysfunction strongly affects the physiology of these cells (Chen et al., Front. Cell Dev. Biol., Sec. Cell Growth and Division, 28 May 2021; 9). AMD resulting from impact trauma impairs mitochondrial activity in chondrocytes, disrupting important processes such as modulation of reactive oxygen species (ROS) and mitochondria-mediated apoptosis (Kan et al., Cartilage. 2021; 13(2 Suppl):1102S-1121S). For example, mitochondrial depolarization and mitochondrial respiratory dysfunction are implicated in AMD. See, e.g., Mckinley et al., Journal of Orthopaedic Trauma 2010; 24:567-570, Huser & Davies, Arthritis Rheum. 2007 July 56(7):2322-34, Delco et al., J Orthop Res. 2018 February 36(2):739-750, Goetz et al., J Orthop Res. 2017 March 35(3):590-599, and Coleman et al., Arthritis Rheumatol. 2016 March 68(3):662-71. Consequently, excessive levels of inflammation and reactive oxygen species (ROS) are typically observed near the site of the injury, which can trigger apoptosis. See, e.g., Wolff et al., J Orthop Res. 2013 Feb; 31(2):191-6, Beecher et al., Iowa Orthop J. 2007; 27:1-8, Brouillette et al., Biomech Model Mechanobiol. 2014 Jun; 13(3):565-72, Lima et al., Osteoarthr Cartil 2001; 9:712-719, and Kurz et al., Arthritis Rheum. 2004 January 50(1):123-30.

In addition to activation of the inflammatory response, various molecular signalling events have been identified in chondrocytes following cartilage injury, including altered gene expression and activation of degradative enzymes, such as matrix metalloproteases (MMPs) (Kramer et al., Int J Clin Exp Med 2011; 4:285-298). Such responses are persistent, contributing feedback loop with destructive consequences for cartilage. In some examples, studies of synovial fluid show abnormalities in GAGs, lubricin, and collagen II, even beyond 12 months post-injury. See, e.g., Wei et al., 2010; J Orthop Res 28:900-906 and Yang et al., Tissue Eng 2006; 12:2957-2964. Decreased activity of AMP-activated protein kinase (AMPK) and the Sirtuin1 (SIRT1) enzyme is also implicated in the acute and/or sustained cellular injury response. See, e.g., Karnik et al., Int J Mol Sci. 2023 March 30; 24(7):6521, Feng et al., Aging (Albany NY). 2020 January 31; 12(2):1087-1103, and Wang et al., Front. Pharmacol., Sec. Experimental Pharmacology and Drug Discovery, 24 July 2020; 11. Together, these damaging responses to mechanical insult affect the entire joint, culminating in PTOA.

The vigorous inflammatory response occurs soon after joint injury and is sustained at lower levels post-injury (Lieberthal et al., Osteoarthritis Cartilage. 2015 Nov; 23(11):1825-1834). Impact-induced matrix degradation and suppressed matrix deposition is also expected in the days and weeks following cartilage injury (Natoli et al., Ann Biomed Eng. 2008 May; 36(5):780-92). Although subtle differences between the development of OA in joints exists, clinical diagnosis generally involves imaging techniques, such as MRI, e.g., to examine evidence of cartilage damage, tissue loss, joint-space narrowing, and the presence of osteophytes. Diagnosis may also involve determining the presence of biomarkers. See, e.g., Harkey et al., Osteoarthritis & Cartilage. 2015; 23(1):1-12. In addition, a clinician may assess pain associated with the affected joint and analyze gait for further insight. See, e.g., Carbone & Rodeo, J Orthop Res. 2017 March 35(3):397-405 and Luyten et al., Knee Surg. Sports Traumatol. Arthrosc. 2012; 20, 401-406.

Additionally, histological scoring systems, such as the Osteoarthritis Cartilage Histopathology (OACH) assessment, the Mankin Histological-Histochemical Grading System (HHGS), and the Osteoarthritis Research Society International (OARSI) scale may provide a quantitative quality to the diagnosis and gradation of severity of OA, including PTOA. See, e.g., Pearson et al., Osteoarthritis Cartilage 2011; 19, 324-331 and Kraus et al., Osteoarthritis Cartilage. 2015 Aug; 23(8):1233-1241. Exemplary anatomical features of OA and PTOA include cartilage and soft tissue degradation, the presence of osteophytes, and visualization of synovial inflammation.

As exemplified by the barbiturate derivative amobarbital, the progression of PTOA can be blocked by agents that rescue the AMD-mediated mitochondrial dysfunction. In particular, intraarticular sodium amytal (amobarbital) blocks mitochondrial electron transport at complex I to prevent chondrocyte dysfunction and apoptosis (Coleman et al., Sci Transl Med. 2018 February 7; 10(427):eaan 5372). However, due to its sedative-hypnotic effects and addictive qualities, clinical application of amobarbital may be limited. Additionally, amobarbital has not been shown to treat the chronic RME that may persist after a joint injury, further underscoring the necessity of providing improved therapeutics for preventing PTOA.

Various lines of evidence, including prospective studies, indicate efficacy of metformin in the context of preventing OA. See, e.g., the prospective study in obese persons by Wang et al., Arthritis Res Ther 20219; 21:127, experimental studies implicating metformin's activation of AMPK and SIRT1, among others, as part of its mechanism of action, e.g., Feng et al., Aging (Albany, NY). 2020 January 31; 12(2):1087-1103, He et al., Cells. 2022 September 27; 11(19):3012, Li et al., Ann Rheum Dis. 2020 May; 79(5):635-645, Lim et al., Osteoarthritis Cartilage. 2022 November 30(11):1434-1442, and Wang et al., Front. Pharmacol., Sec. Experimental Pharmacology and Drug Discovery, 24 July 2020; 11, and reviews describing the various beneficial effects of metformin, including modulation of inflammation, oxidative stress, autophagy, adipokine levels, and the microbiome, by Song et al., Front Pharmacol. 2022 August 23; 13:952560 and Lambova et al., Life (Basel). 2023 February 3; 13(2):437.

However, studies to date have not examined the efficacy of intraarticular metformin as a treatment in traumatic joint injury that includes AMD. In contrast to models involving slow degenerative injury, acute direct cartilage damage may be likened to a fracture or major ligament injury. Additionally, surgically-induced models of trauma, such as the destabilization of the medial meniscus (DMM) model fail to overload or damage cartilage, as is characteristic of joint trauma. For example, in mouse and non-human primate models of surgically induced joint instability, which mimics the RME that can persist after joint trauma but does not include AMD from a traumatic overload, metformin has been shown to be effective at preventing or delaying the onset of OA. See, e.g., Feng et al., Aging (Albany NY). 2020 January 16; 12(2):1087-1103, Li et al., Ann Rheum Dis. 2020 May; 79(5):635-645, Li et al., Arthritis Res Ther. 2020 February 22; 22(1):34, and Wang et al., Front. Pharmacol., Sec. Experimental Pharmacology and Drug Discovery, 24 July 2020; 11. Metformin has been studied in other rodent models of OA, including those where joint degeneration was induced by collagenase or by metabolism inhibitor iodoacetate, and in ovariectomized mice that were fed a high-fat diet. See, e.g., Belenska-Todorova et al., Biomedicines. 2021 Aug; 9(8):1017, Li et al., Bone. 2022 April 157:116323, Fan et al., Ann Transl Med. 2020 Dec. 8(23):1565, and Na et al., Cells. 2021 March 19; 10(3):681. Regardless, such studies are silent on the safety and efficacy of administering metformin via the intraarticular route, especially at the levels explored herein, in the context of preventing AMD-initiated PTOA, a deficiency cured by the disclosed methods.

Furthermore, no studies known to the present inventors have explored the effects of relatively high concentrations of intraarticular metformin, for example, at levels of 50 mM or more. Metformin is commonly prescribed as an anti-diabetic treatment for oral administration, and side effects include nausea, vomiting, diarrhoea, and stomach pain. Maximum oral doses of metformin in the treatment of diabetes range from 2,500 mg to 4,500 mg (1,500 mg take three times per day) (Das et al., Clin Med Insights Endocrinol Diabetes. 2021; 14:11795514211030513 and Kant et al., J Diabetes Investig. 2018 May; 9(3):587-593). However, studying the pharmacokinetics of oral metformin revealed an inverse relationship between dose and bioavailability, i.e., lower doses are associated with greater bioavailability than higher doses. See, e.g., Scheen, Clin. Pharmacokinet. 1996 May; 30 (5); 359-371 and Tucker et al., Br J Clin Pharmacol. 1981 Aug. 12(2):235-246. As the studies described herein show that relatively high concentrations of metformin are necessary to rescue chondrocyte function in impacted joints, even such high oral doses may be insufficient to treat an injured joint and prevent the development of PTOA. See, e.g., Example 1 herein. Routes of administration may not be interchangeable due to volume, dosage, excipients, and other factors known to those of skill in the art. Therefore, the methods described herein are distinct from uses of metformin consisting only of oral administration. As disclosed herein, metformin administered by the intraarticular route, injected directly into the joint space, provides clinically effective dosage to rescue chondrocyte function. Additionally, as described herein, it is not merely just a dose-dependent increase, but a specific dosing range that provides the therapeutic benefit. See, e.g., Example 3. Disclosed herein are methods and compositions for overcoming the limitations of dosing metformin to treat injured joints.

Grammatical variations of “administer,” “administration,” and “administering” to a subject include any route of introducing or delivering to a subject an agent. Administration can be carried out by any suitable route, including oral, topical, intravenous, subcutaneous, transcutaneous, transdermal, intramuscular, intra-joint, intraarticular, parenteral, intra-arteriole, intradermal, intraventricular, intracranial, intraperitoneal, intralesional, intranasal, rectal, vaginal, by implanted reservoir, and the like.

Herein, the term “pharmaceutically acceptable salt” refers to salts prepared from pharmaceutically acceptable non-toxic acids or bases, and which may be synthesized by conventional chemical methods. Exemplary salts of metformin include but are not limited to phosphate salts, sulfate salts, hydrochloride salt, salicylate salts, maleate salts, benzoate salts, ethanedisulfonate salts, fumarate salts, glycolate salts, succinate salts (2:1 molar ratio of metformin: dibasic acid), docusate salt, and decavanadate salt. Pharmaceutical acceptability of the metformin salt can be determined by evaluating solubility, stability, e.g., whether the salt has increased solubility and stability compared to the metformin base form, hygroscopicity, flow, compaction susceptibility, polymorphism, counterion effects, and the like.

Herein “treating,” “treatment,” “preventing,” “prevention,” “inhibiting,” “inhibition,” “reducing,” “reduction,” and the like, with reference to post-traumatic osteoarthritis (PTOA), refer to achieving a desired therapeutic result, such as mitigating and preventing the development of PTOA. Preventing the development of PTOA may involve any of a reduction, delay, or absence of development of one or more symptoms of PTOA. Exemplary symptoms of PTOA include joint pain, joint stiffness, loss of tissue, such as cartilage degeneration, and inflammation. Accordingly, desired therapeutic results include reduction and/or prevention of joint pain, joint stiffness, loss of tissue, such as cartilage degeneration, and inflammation. Additional beneficial therapeutic effects of the methods and compositions described herein will be evident to one of skill in the art.

Factors contributing to the initiation and development of the disease state include, e.g., mitochondrial dysfunction in chondrocytes, reduced viability of chondrocytes, increased inflammation, such as evidenced by greater levels ROS, enhanced apoptosis, and cartilage matrix disruption post-impact. These and other contributory factors known to one of skill in the art may be observed in vitro or ex vivo, e.g., from analysis of explanted tissue.

### Method of Treatment

In some aspects, provided herein are methods of treating injured joints in a mammal comprising administering metformin or a pharmaceutically acceptable salt thereof via intraarticular injection to the injured joint. In some embodiments, administering metformin or a pharmaceutically acceptable salt thereof via intraarticular injection to the injured joint, such as treating the injured joint, prevents the pathogenesis of osteoarthritis (OA), such as post-traumatic osteoarthritis (PTOA). In some embodiments, the injured joint is a knee joint, a hip joint, an ankle joint, or an elbow joint. In some embodiments, the injury is any of a fracture, cartilage damage, acute ligament sprain, chronic ligamentous instability, or a combination of the same. In some embodiments, metformin hydrochloride or the synonym 1,1-dimethylbiguanide hydrochloride. In preferred embodiments, the mammal is human.

In some embodiments, administering metformin or a pharmaceutically acceptable salt thereof via intraarticular injection to an injured joint treats the injured joint in a mammal. In some embodiments, administering metformin or a pharmaceutically acceptable salt thereof via intraarticular injection to an injured joint in combination with systemic administration of metformin or a pharmaceutically acceptable salt thereof treats the injured joint in a mammal. In some embodiments, administering metformin or a pharmaceutically acceptable salt thereof via intraarticular injection to an injured joint in combination with oral administration of metformin or a pharmaceutically acceptable salt thereof treats the injured joint in a mammal. In some embodiments, administering metformin or a pharmaceutically acceptable salt thereof via intraarticular injection to an injured joint in combination with intravenous (IV) administration of metformin or a pharmaceutically acceptable salt thereof treats the injured joint in a mammal. In some embodiments, administering metformin or a pharmaceutically acceptable salt thereof via intraarticular injection to an injured joint in combination with intramuscular (IM) administration of metformin or a pharmaceutically acceptable salt thereof treats the injured joint in a mammal. In some embodiments, administering metformin or a pharmaceutically acceptable salt thereof via intraarticular injection to an injured joint in combination with any one or more of oral, IV, and IM administration of metformin or a pharmaceutically acceptable salt thereof treats the injured joint in a mammal.

In some embodiments, treating an injured joint in a mammal comprises administering metformin or a pharmaceutically acceptable salt thereof via the intraarticular route to the mammal at a concentration of about 50 mM to 150 mM, 50 mM to 125 mM, 50 mM to 120 mM, 50 mM to 115 mM, 50 mM to 110 mM, 50 mM to 105 mM, 50 mM to 100 mM, 50 mM to 95 mM, 50 mM to 90 mM, 50 mM to 85 mM, 50 mM to 80 mM, 50 mM to 75 mM, 50 mM to 70 mM, 50 mM to 65 mM, 50 mM to 60 mM, or 50 mM to 55 mM, wherein each range is inclusive.

In some embodiments, treating an injured joint in a mammal comprises administering metformin or a pharmaceutically acceptable salt thereof via the intraarticular route to the mammal at a concentration of about 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, 100 mM, 105 mM, 110 mM, 115 mM, 120 mM, 125 mM, 130 mM, 135 mM, 140 mM, 145 mM, or 150 mM.

In some embodiments, treating an injured joint in a mammal comprises administering metformin or a pharmaceutically acceptable salt thereof via the intraarticular route in combination with a viscosupplement. In some embodiments, the viscosupplement comprises hyaluronic acid. In some embodiments, the hyaluronic acid is delivered in the form of a hydrogel. In some embodiments, the hydrogel comprises hyaluronic acid and metformin.

In some embodiments, treating an injured joint in a mammal further comprises administering an additional agent, such as an analgesic, a chondroprotective agent, a growth factor, a hormone, a hyaluronate, a non-steroidal anti-inflammatory drug (NSAID), a steroid, a vitamin, or any combination of the same. In some embodiments, the additional agent is delivered via intraarticular injection. In some embodiments, treating an injured joint in a mammal further comprises providing physical rehabilitation and therapy.

In some examples, treating an injured joint comprises reducing inflammation relative to pre-treatment levels, such as levels of reactive oxygen species (ROS), and preserving the viability and function of mitochondria and chondrocytes in the impacted or injured cartilage.

In some examples, preventing PTOA comprises preventing tissue loss and matrix disruption, e.g., as determined by imaging techniques and standardized measures of articular cartilage damage, such as the OARSI scoring system. In the clinic, the current standard remains the Kellgren Lawrence Score. See, e.g., Holzer et al., Osteoarthritis Cartilage, 2015. 23(3): p. 363-9. An additional method for detecting tissue loss is weight-bearing CT scanning, such as described by Willey et al., J Bone Joint Surg Am, 2020. 102(9): p. 796-803.

In some embodiments, treatment according the disclosed methods prevents changes in histopathology scores relative to pre-treatment scores that are indicative of disease progression. Such determinations are within the grasp of one of skill in the art. For example, using the OARSI scoring system, the lowest score is indicative of intact normal tissue (Grade 0), and higher scores are indicative of disease progression, e.g., uneven but intact surface (Grade 1), surface discontinuity (Grade 2), vertical fissures (Grade 3), erosion (Grade 4), and denudation (Grade 5). Cartilage with no signs of degeneration (OARSI Grade 0) has significantly better biomechanical properties than cartilage with signs of early degeneration (OARSI Grade 1), and cartilage with more advanced degeneration according to the OARSI system has a reduced ability to withstand compressive loads. See, e.g., Pritzker et al., Osteoarthritis & Cartilage 14:13-29 and Waldstein et al., J Orthop Res. 2016 January 34(1):135-40.

In some embodiments, treatment according the disclosed methods prevents observances in imaging analysis, e.g., MRI, radiograph, weight-bearing CT scans, and related techniques, that are indicative of disease progression. For example, OA may be graded radiographically using the Kellgren-Lawrence (K-L) scale to detect the presence of, e.g., cartilage and bone marrow lesions, osteophytes, and effusion. See, e.g., Kellgren & Lawrence, Ann Rheum Dis. 1957; 16 4:494-502, Holzer et al., Osteoarthritis Cartilage. 2015Mar. 23 (3):363-9, and Park et al., Eur J Radiol. 2013 January 82(1):112-7. The K-L scale ranges from 0 to 4 based on the presence and degree of osteophytes, joint-space narrowing, sclerosis, and deformity, with grades of 2 or higher indicating the presence of radiographic OA. In some embodiments, treatment of injured joints according to the present disclosure prevents increases in K-L scores relative to pre-treatment scores, which are indicative of disease progression.

Additional observable determinants of OA/PTOA disease progression, which may be prevented by treating a subject in accordance with the methods disclosed herein, include altered gait analysis and biomarkers. Determining the prevention of PTOA according to these elements are available to one of skill in the art. For example, regarding gait analysis, the progression of OA is associated with larger heel-strike knee flexion angle (KFA) and larger mid-stance knee adduction moment (KAM) and knee flexion moment (KFM) (Favre & Jolles, EFORT Open Rev 2016; 1:368-374).

In some embodiments, treatment according to the disclosed methods prevents or reduces the level of biomarkers associated with PTOA. In some examples, biomarkers may be examined in the synovial fluid of the injured joint. See, e.g., Carter et al., Biomech. 2015; 48 8: 1461-1468 and Harkey et al., Osteoarthritis Cartilage. 2015 Jan; 23(1):1-12. Determining PTOA by biomarkers may include assessment of cartilage oligomeric matrix protein, inflammatory cytokines, matrix metalloproteinase (MMP) activity, prostaglandin E2, sulfated glycosaminoglycan, and elevated collagen turnover, determined, e.g., by measuring collagen type II cleavage biomarker C2C.

### Pain

In some aspects, provided herein are methods of preventing or reducing the symptoms of OA, such as PTOA, thereby inhibiting the initiation and/or development of PTOA, e.g., by preventing pain associated with an injured joint from worsening and/or reducing pain in a mammal with an injured joint comprising administering a pharmaceutically acceptable salt of metformin via intraarticular injection to the injured joint. In some embodiments, administering metformin or a pharmaceutically acceptable salt thereof via intraarticular injection to the injured joint prevents pain associated with osteoarthritis (OA), such as post-traumatic osteoarthritis (PTOA). In some embodiments, the pharmaceutically acceptable salt of metformin is metformin hydrochloride.

Herein, “preventing pain from worsening” may be referred to simply as “preventing pain.” In some embodiments, reductions in pain include reductions in any one or more of aching, stiffness, and disability. In some embodiments, the injured joint is a wrist joint, a shoulder joint, a knee joint, a hip joint, an ankle joint, or an elbow joint. In some embodiments, the injury is any of a fracture, cartilage damage, acute ligament sprain, chronic ligamentous instability, or a combination of the same. In preferred embodiments, the mammal is human.

In some embodiments, administering metformin or a pharmaceutically acceptable salt thereof via intraarticular injection to a mammal with an injured joint prevents or reduces pain associated with the injury. In some embodiments, administering metformin or a pharmaceutically acceptable salt thereof via intraarticular injection to an injured joint in combination with systemic administration of metformin or a pharmaceutically acceptable salt thereof prevents pain associated with the injury from worsening and/or reduces pain associated with the injury. In some embodiments, administering metformin or a pharmaceutically acceptable salt thereof via intraarticular injection to an injured joint in combination with oral administration of metformin prevents pain associated with the injury from worsening and/or reduces pain associated with the injury. In some embodiments, administering metformin or a pharmaceutically acceptable salt thereof via intraarticular injection to an injured joint in combination with intravenous (IV) administration of metformin or a pharmaceutically acceptable salt thereof prevents or reduces pain associated with the injury. In some embodiments, administering metformin or a pharmaceutically acceptable salt thereof via intraarticular injection to an injured joint in combination with intramuscular (IM) administration of metformin prevents pain associated with the injury from worsening and/or reduces pain associated with the injury. In some embodiments, administering metformin or a pharmaceutically acceptable salt thereof via intraarticular injection to an injured joint in combination with any one or more of oral, IV, and IM administration of metformin prevents pain associated with the injury from worsening and/or reduces pain associated with the injury.

In some embodiments, preventing or reducing the pain of an injured joint in a mammal comprises administering metformin or a pharmaceutically acceptable salt thereof via the intraarticular route to the mammal at a concentration of about 50 mM to 150 mM, 50 mM to 125 mM, 50 mM to 120 mM, 50 mM to 115 mM, 50 mM to 110 mM, 50 mM to 105 mM, 50 mM to 100 mM, 50 mM to 95 mM, 50 mM to 90 mM, 50 mM to 85 mM, 50 mM to 80 mM, 50 mM to 75 mM, 50 mM to 70 mM, 50 mM to 65 mM, 50 mM to 60 mM, or 50 mM to 55 mM, wherein each range is inclusive.

In some embodiments, preventing or reducing the pain of an injured joint in a mammal comprises administering metformin or a pharmaceutically acceptable salt thereof or a pharmaceutically acceptable salt thereof via the intraarticular route to the mammal at a concentration of about 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, 100 mM, 105 mM, 110 mM, 115 mM, 120 mM, or 125 mM.

In preferred embodiments, preventing or reducing the pain of an injured joint in a mammal comprises administering metformin or a pharmaceutically acceptable salt thereof via the intraarticular route to the mammal at a concentration of about 40 mM, 45 mM, 50 mM, 55 mM, 60 mM, 65 mM, 70 mM, 75 mM, 80 mM, 85 mM, 90 mM, 95 mM, or 100 mM.

In some embodiments, preventing or reducing the pain of an injured joint in a mammal comprises administering metformin or a pharmaceutically acceptable salt thereof via the intraarticular route in combination with a viscosupplement. In some embodiments, the viscosupplement comprises hyaluronic acid. In some embodiments, the hyaluronic acid is delivered in the form of a hydrogel. In some embodiments, the hydrogel comprises hyaluronic acid and metformin or a pharmaceutically acceptable salt thereof.

In some embodiments, preventing or reducing the pain of an injured joint in a mammal further comprises administering an additional pain relief agent. In some embodiments, the additional pain relief agent is an analgesic, an anticonvulsant, an antipyretic, a corticosteroid, a non-steroidal anti-inflammatory drug (NSAID), an opiate, an opioid, a serotonin norepinephrine reuptake inhibitor (SNRI), a tricyclic antidepressant (TCA) or any combination thereof.

In some embodiments, treatment according to disclosed methods prevents OA/PTOA symptoms, such as joint pain and/or joint stiffness. In some embodiments, treatment according to disclosed methods reduces pain associated with an injury. In some embodiments, preventing pain from worsening and/or reducing pain are determined in a subject relative to pain levels before treatment with metformin or a pharmaceutically acceptable salt thereof via IA injection, according to the methods described herein. Determinations of pain prevention and pain reduction are available to one of skill in the art. See, e.g., van Laar et al., Open Rheumatol J. 2012; 6:320-330. For example, pain prevention and/or pain reduction may be determined by a general self-reported scale, such as the Visual Analog Scale (VAS). Alternatively, symptoms, such as pain, stiffness, and disability, may be evaluated using joint-specific measures.

For example, the Western Ontario and McMaster Universities Arthritis Index (WOMAC) is a widely used self-reported evaluation of hip and knee osteoarthritis. The WOMAC evaluates pain (5 items): during walking, using stairs, in bed, sitting or lying, and standing upright; stiffness (2 items): after first waking and later in the day; and physical function (17 items): using stairs, rising from sitting, standing, bending, walking, getting in/out of a car, shopping, putting on/taking off socks, rising from bed, lying in bed, getting in/out of bath, sitting, getting on/off toilet, heavy domestic duties, light domestic duties.

### Dosing

In some aspects, provided are dosing regimens effective to prevent OA, such as PTOA. In some embodiments, the methods described herein comprise administering an injectable composition comprising a pharmaceutically acceptable salt of metformin thereof via the intraarticular route to an injured joint in a mammal at a concentration of up to and including about 85 mM, 90 mM, 95 mM, 100 mM, 105 mM, 110 mM, 115 mM, 120 mM, 125 mM, 130 mM, 135 mM, 140 mM, 145 mM, 150 mM, 155 mM, 160 mM, 165 mM, 170 mM, 175 mM, 180 mM, 185 mM, 190 mM, 195 mM, or 200 mM. In some embodiments, the methods described herein comprise administering the pharmaceutically acceptable salt of metformin via the intraarticular route at a concentration of up to and including about 100 mM, 105 mM, 110 mM, 115 mM, 120 mM, 125 mM, 130 mM, 135 mM, 140 mM, 145 mM, or 150 mM. In some embodiments, the methods described herein comprise administering the pharmaceutically acceptable salt of metformin via the intraarticular route at a concentration of less than about 100 mM, 105 mM, 110 mM, 115 mM, 120 mM, 125 mM, 130 mM, 135 mM, 140 mM, 145 mM, or 150 mM.

In some embodiments, the methods described herein comprise administering the pharmaceutically acceptable salt of metformin via the intraarticular route to the injured joint in the mammal at a concentration of about 25 mM to 150 mM, 25 mM to 125 mM, 25 mM to 120 mM, 25 mM to 115 mM, 25 mM to 110 mM, 25 mM to 105 mM, 25 mM to 100 mM, 25 mM to 95 mM, 25 mM to 90 mM, 25 mM to 85 mM, 25 mM to 80 mM, 25 mM to 75 mM, 25 mM to 70 mM, 25 mM to 65 mM, 25 mM to 60 mM, 25 mM to 55 mM, 25 mM to 50 mM, 25 mM to 45 mM, or 25 mM to 40 mM, wherein each range is inclusive.

In some embodiments, the methods described herein comprise administering the pharmaceutically acceptable salt of metformin via the intraarticular route to the injured joint in the mammal at a concentration of about 30 mM to 150 mM, 30 mM to 125 mM, 30 mM to 120 mM, 30 mM to 115 mM, 30 mM to 110 mM, 30 mM to 105 mM, 30 mM to 100 mM, 30 mM to 95 mM, 30 mM to 90 mM, 30 mM to 85 mM, 30 mM to 80 mM, 30 mM to 75 mM, 30 mM to 70 mM, 30 mM to 65 mM, 30 mM to 60 mM, 30 mM to 55 mM, 30 mM to 50 mM, 30 mM to 45 mM, or 30 mM to 40 mM, wherein each range is inclusive.

In some embodiments, the methods described herein comprise administering the pharmaceutically acceptable salt of metformin via the intraarticular route to the injured joint in the mammal at a concentration of about 35 mM to 150 mM, 35 mM to 125 mM, 35 mM to 120 mM, 35 mM to 115 mM, 35 mM to 110 mM, 35 mM to 105 mM, 35 mM to 100 mM, 35 mM to 95 mM, 35 mM to 90 mM, 35 mM to 85 mM, 35 mM to 80 mM, 35 mM to 75 mM, 35 mM to 70 mM, 35 mM to 65 mM, 35 mM to 60 mM, 35 mM to 55 mM, 35 mM to 50 mM, 35 mM to 45 mM, or 35 mM to 40 mM, wherein each range is inclusive.

In some embodiments, the methods described herein comprise administering the pharmaceutically acceptable salt of metformin via the intraarticular route to the injured joint in the mammal at a concentration of about 40 mM to 150 mM, 40 mM to 125 mM, 40 mM to 120 mM, 40 mM to 115 mM, 40 mM to 110 mM, 40 mM to 105 mM, 40 mM to 100 mM, 40 mM to 95 mM, 40 mM to 90 mM, 40 mM to 85 mM, 40 mM to 80 mM, 40 mM to 75 mM, 40 mM to 70 mM, 40 mM to 65 mM, 40 mM to 60 mM, 40 mM to 55 mM, 40 mM to 50 mM, or 40 mM to 45 mM metformin, wherein each range is inclusive.

In some embodiments, the methods described herein comprise administering the pharmaceutically acceptable salt of metformin via the intraarticular route to the injured joint in the mammal at a concentration of about 45 mM to 150 mM, 45 mM to 125 mM, 45 mM to 120 mM, 45 mM to 115 mM, 45 mM to 110 mM, 45 mM to 105 mM, 45 mM to 100 mM, 45 mM to 95 mM, 45 mM to 90 mM, 45 mM to 85 mM, 45 mM to 80 mM, 45 mM to 75 mM, 45 mM to 70 mM, 45 mM to 65 mM, 45 mM to 60 mM, 45 mM to 55 mM, or 45 mM to 50 mM, wherein each range is inclusive.

In some embodiments, the methods described herein comprise administering the pharmaceutically acceptable salt of metformin via the intraarticular route to the injured joint in the mammal at a concentration of about 50 mM to 150 mM, 50 mM to 125 mM, 50 mM to 120 mM, 50 mM to 115 mM, 50 mM to 110 mM, 50 mM to 105 mM, 50 mM to 100 mM, 50 mM to 95 mM, 50 mM to 90 mM, 50 mM to 85 mM, 50 mM to 80 mM, 50 mM to 75 mM, 50 mM to 70 mM, 50 mM to 65 mM, 50 mM to 60 mM, or 50 mM to 55 mM, wherein each range is inclusive.

In some embodiments, the methods described herein comprise administering the pharmaceutically acceptable salt of metformin via the intraarticular route to the injured joint in the mammal at a concentration of about 55 mM to 150 mM, 55 mM to 125 mM, 55 mM to 120 mM, 55 mM to 115 mM, 55 mM to 110 mM, 55 mM to 105 mM, 55 mM to 100 mM, 55 mM to 95 mM, 55 mM to 90 mM, 55 mM to 85 mM, 55 mM to 80 mM, 55 mM to 75 mM, 55 mM to 70 mM, 55 mM to 65 mM, or 55 mM to 60 mM, wherein each range is inclusive.

In some embodiments, the methods described herein comprise administering the pharmaceutically acceptable salt of metformin via the intraarticular route to the injured joint in the mammal at a concentration of about 60 mM to 150 mM, 60 mM to 125 mM, 60 mM to 120 mM, 60 mM to 115 mM, 60 mM to 110 mM, 60 mM to 105 mM, 60 mM to 100 mM, 60 mM to 95 mM, 60 mM to 90 mM, 60 mM to 85 mM, 60 mM to 80 mM, 60 mM to 75 mM, 60 mM to 70 mM, or 60 mM to 65 mM, wherein each range is inclusive.

In some embodiments, the methods described herein comprise administering the pharmaceutically acceptable salt of metformin via the intraarticular route to the injured joint in the mammal at a concentration of about 65 mM to 150 mM, 65 mM to 125 mM, 65 mM to 120 mM, 65 mM to 115 mM, 65 mM to 110 mM, 65 mM to 105 mM, 65 mM to 100 mM, 65 mM to 95 mM, 65 mM to 90 mM, 65 mM to 85 mM, 65 mM to 80 mM, 65 mM to 75 mM, or 65 mM to 70 mM, wherein each range is inclusive.

In some embodiments, the methods described herein comprise administering the pharmaceutically acceptable salt of metformin, such as metformin hydrochloride, via the intraarticular route to the injured joint in the mammal at a concentration of about 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, 100 mM, 105 mM, 110 mM, 115 mM, 120 mM, 125 mM, 130 mM, 135 mM, 140 mM, 145 mM, 150 mM, 155 mM, 160 mM, 165 mM, 170 mM, 175 mM, 180 mM, 185 mM, 190 mM, 195 mM, or 200 mM. In preferred embodiments, the pharmaceutically acceptable salt of metformin is metformin hydrochloride.

In other preferred embodiments, the methods described herein comprise administering pharmaceutically acceptable salt of metformin hydrochloride via the intraarticular route to the injured joint in the mammal at a concentration of about 40 mM, 45 mM, 50 mM, 55 mM, 60 mM, 65 mM, 70 mM, 75 mM, 80 mM, 85 mM, 90 mM, 95 mM, or 100 mM.

In some embodiments, the injectable composition comprising the pharmaceutically acceptable salt of metformin is administered via intraarticular injection to the injured joint in the mammal in a volume of about 0.5 to 15 mL, 0.5 to 10 mL, 0.5 to 5 mL, 1 to 15 mL, 1 to 10 mL, 1 to 5 mL, 2 to 15 mL, 2 to 10 mL, 2 to 5 mL, 3 to 15 mL, 3 to 10 mL, 3 to 5 mL, 4 to 15 mL, 4 to 10 mL, or 4 to 5 mL wherein each range is inclusive. In some embodiments, metformin is administered via intraarticular injection in a volume of 0.1 mL, 0.25 mL, 0.5 mL, 0.75 mL, 1 mL, 2 mL, 3 mL, 4 mL, 5 mL, 6 mL, 7 mL, 8 mL, 9 mL, 10 mL, 11 mL, 12 mL, 13 mL, 14 mL, or 15 mL. In preferred embodiments, the volume of the injectable metformin composition administered via intraarticular injection is 2 to 3 mL.

In some embodiments, the injectable composition comprising metformin or a pharmaceutically acceptable salt thereof is administered via intraarticular injection to an injured joint in a mammal only once. In some embodiments, metformin is administered via intraarticular injection to an injured joint in a mammal more than one time. In some embodiments, metformin is administered via intraarticular injection about every hour, every three hours, every six hours, every nine hours, every 12 hours, daily, every other day, once a week, twice a week, three times a week, four times a week, once every two weeks, once every three weeks, once every four weeks, once a month, once every two months, once every three months, or once every four months.

In some embodiments, metformin or a pharmaceutically acceptable salt thereof is administered via intraarticular injection over the course of one day, two days, three days, four days, five days, six days, up to one week, up to two weeks, up to three weeks, up to four weeks, up to five weeks, up to six weeks, up to seven weeks, up to eight weeks, up to nine weeks, up to 10 weeks, up to one month, up to two months, up to three months, up to four months, up to five months, up to six months, up to seven months, up to 8 months, up to nine months, up to 10 months, up to 11 months, or up to 12 months, wherein each range is inclusive.

In some embodiments, disclosed methods comprise administering metformin or a pharmaceutically acceptable salt thereof, such as metformin hydrochloride, to an injured joint in a mammal via intraarticular administration. In some embodiments, metformin salts for use in accordance with disclosed methods include any of metformin benzoate, metformin ethanesulfonate, metformin (2:1) fumarate, metformin glycinate, metformin glycolate, metformin hydrobromide, metformin hydrochloride, metformin maleate, metformin phosphate, metformin salicylate, metformin (2:1) succinate, and metformin sulfate. Such metformin salts are described in BE568513A, U.S. Pat. No. 8,703,183B2, U.S. Pat. No. 6,031,004A, and U.S. Pat. No. 3,174,901A. Additional metformin formulations, which may be used in accordance with the disclosed methods, include fatty acid complexes, e.g., as described in US20050158374A1, lipophilic acid salts, e.g., as described in US20030220301A1, and controlled release formulations, such as described in US20060034922A1 and WO 99/47128. In preferred embodiments, disclosed methods comprise administering an injectable composition comprising metformin hydrochloride by intraarticular injection to an injured joint of a mammal, preferably a human.

Additional pharmaceutically acceptable salts of metformin which may be used in accordance with disclosed methods include the (4-chlorophenoxy) isobutyrate salt (FR2275199A1), acetylsalicylate acid salt (U.S. Pat. No. 3,957,853A), chlorophenoxyacetic acid salt (U.S. Pat. No. 4,835,184A), clofribrate salt (U.S. Pat. No. 4,080,472A), dichloroacetic acid salt (U.S. Pat. No. 4,028,402A), nicotinic acid salt (DE2357864A1), orotate salt (FR2264539A1), pamoate salt (FR2037002A1 and FR2320735A1).

### Intraarticular Administration Combined With Additional Routes of Administration

In some aspects, provided are methods comprising administering metformin or a pharmaceutically acceptable salt thereof via the intraarticular route in combination with systemic administration of metformin, such as administration of a systemic formulation of metformin for preventing OA, such as PTOA. In some embodiments, systemic administration of metformin, such as a pharmaceutically acceptable salt thereof, consists only of oral administration, IM administration, or IV administration. In some embodiments, systemic administration of metformin, such as a pharmaceutically acceptable salt thereof, comprises any combination of oral administration, IM administration, and IV administration.

In some embodiments, in addition to intraarticular administration, the systemic formulation of metformin is administered via a systemic route about every hour, every three hours, every six hours, every nine hours, every 12 hours, daily, every other day, once a week, twice a week, three times a week, four times a week, once every two weeks, once every three weeks, once every four weeks, once a month, once every two months, once every three months, or once every four months.

In some embodiments, the systemic formulation of metformin comprises a pharmaceutically acceptable salt of metformin, such as any of metformin benzoate, metformin ethanesulfonate, metformin (2:1) fumarate, metformin glycinate, metformin glycolate, metformin hydrobromide, metformin hydrochloride, metformin maleate, metformin phosphate, metformin salicylate, metformin (2:1) succinate, and metformin sulfate. In some embodiments, the systemic formulation of metformin comprises metformin hydrochloride. In some embodiments, the systemic formulation of metformin further comprises any one or more of alogliptin, canagliflozin, dapagliflozin, empagliflozin, ertugliflozin, glipizide, glyburide, and linagliptin, including pharmaceutically acceptable salts thereof, e.g., alogliptin benzoate.

In some embodiments, in addition to intraarticular administration, the systemic formulation of metformin, such as a formulation suitable for systemic administration comprising a pharmaceutically acceptable salt of metformin, is systemically administered to a mammal having an injured joint in an amount of at least about 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 1950 mg, 2000 mg, 2050 mg, 2100 mg, 2150 mg, 2200 mg, 2250 mg, 2300 mg, 2350 mg, 2400 mg, 2450 mg, 2500 mg, 2550 mg, 2600 mg, 2650 mg, 2700 mg, 2750 mg, 2800 mg, 2850 mg, 2900 mg, 2950 mg, or 3000 mg per day.

In some embodiments, the systemic formulation of metformin is suitable for oral administration. In some embodiments, the systemic formulation of metformin is suitable for IM administration. In some embodiments, the systemic formulation of metformin is suitable for IV administration.

In some embodiments, in addition to intraarticular administration, metformin or a pharmaceutically acceptable salt thereof is orally administered to a mammal having an injured joint in an amount of up to and including about 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 1950 mg, 2000 mg, 2050 mg, 2100 mg, 2150 mg, 2200 mg, 2250 mg, 2300 mg, 2350 mg, 2400 mg, 2450 mg, 2500 mg, 2550 mg, 2600 mg, 2650 mg, 2700 mg, 2750 mg, 2800 mg, 2850 mg, 2900 mg, 2950 mg, or 3000 mg per day. In preferred embodiments, the pharmaceutically acceptable salt of metformin is metformin hydrochloride.

In some embodiments, in addition to intraarticular administration, metformin or a pharmaceutically acceptable salt thereof is orally administered to a mammal having an injured joint in an amount of about 100 mg to 6000 mg, 200 mg to 6000 mg, 300 mg to 6000 mg, 400 mg to 6000 mg, 500 mg to 6000 mg, 600 mg to 6000 mg, 700 mg to 6000 mg, 800 mg to 6000 mg, 900 mg to 6000 mg, or 1000 mg to 6000 mg per day, wherein each range is inclusive.

In some embodiments, in addition to intraarticular administration, metformin or a pharmaceutically acceptable salt thereof is orally administered to a mammal having an injured joint in an amount of about 100 mg to 5000 mg, 200 mg to 5000 mg, 300 mg to 5000 mg, 400 mg to 5000 mg, 500 mg to 5000 mg, 600 mg to 5000 mg, 700 mg to 5000 mg, 800 mg to 5000 mg, 900 mg to 5000 mg, or 1000 mg to 5000 mg per day, wherein each range is inclusive.

In some embodiments, in addition to intraarticular administration, metformin or a pharmaceutically acceptable salt thereof is orally administered to a mammal having an injured joint in an amount of about 100 mg to 4000 mg, 200 mg to 4000 mg, 300 mg to 4000 mg, 400 mg to 4000 mg, 500 mg to 4000 mg, 600 mg to 4000 mg, 700 mg to 4000 mg, 800 mg to 4000 mg, 900 mg to 4000 mg, or 1000 mg to 4000 mg per day, wherein each range is inclusive.

In some embodiments, in addition to intraarticular administration, metformin or a pharmaceutically acceptable salt thereof is orally administered to a mammal having an injured joint in an amount of about 100 mg to 3000 mg, 200 mg to 3000 mg, 300 mg to 3000 mg, 400 mg to 3000 mg, 500 mg to 3000 mg, 600 mg to 3000 mg, 700 mg to 3000 mg, 800 mg to 3000 mg, 900 mg to 3000 mg, or 1000 mg to 3000 mg per day, wherein each range is inclusive.

In some embodiments, in addition to intraarticular administration, metformin or a pharmaceutically acceptable salt thereof is orally administered to a mammal having an injured joint in an amount of about 100 mg to 2000 mg, 200 mg to 2000 mg, 300 mg to 2000 mg, 400 mg to 2000 mg, 500 mg to 2000 mg, 600 mg to 2000 mg, 700 mg to 2000 mg, 800 mg to 2000 mg, 900 mg to 2000 mg, or 1000 mg to 2000 mg per day, wherein each range is inclusive.

In some embodiments, in addition to intraarticular administration, metformin or a pharmaceutically acceptable salt thereof is orally administered to a mammal having an injured joint in an amount of about 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 1950 mg, 2000 mg, 2050 mg, 2100 mg, 2150 mg, 2200 mg, 2250 mg, 2300 mg, 2350 mg, 2400 mg, 2450 mg, 2500 mg, 2550 mg, 2600 mg, 2650 mg, 2700 mg, 2750 mg, 2800 mg, 2850 mg, 2900 mg, 2950 mg, or 3000 mg per day.

In some embodiments, in addition to intraarticular administration, metformin or a pharmaceutically acceptable salt thereof is orally administered to a mammal having an injured joint in an amount of 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, or 1500 mg per day. In preferred embodiments, in addition to intraarticular administration, metformin is orally administered to a mammal having an injured joint in an amount of about 1000 mg per day.

In some embodiments, the administered oral metformin composition comprises a pharmaceutically acceptable salt of metformin, such as metformin hydrochloride. In some embodiments, the administered oral metformin composition further comprises any one or more of alogliptin, canagliflozin, dapagliflozin, empagliflozin, ertugliflozin, glipizide, glyburide, and linagliptin, including pharmaceutically acceptable salts thereof, e.g., alogliptin benzoate.

In some embodiments, in addition to intraarticular administration, metformin or a pharmaceutically acceptable salt thereof is systemically administered via the oral route only once, i.e., a total of one time. In some embodiments, in addition to intraarticular administration, metformin is systemically administered via the oral route more than once. In some embodiments, in addition to intraarticular administration, metformin is administered via the oral route daily, every other day, once a week, twice a week, three times a week, four times a week, once every two weeks, once every three weeks, once every four weeks, once a month, once every two months, once every three months, or once every four months.

In some embodiments, in addition to intraarticular administration, metformin or a pharmaceutically acceptable salt thereof is systemically administered via the intravenous (IV) route only once, i.e. a total of one time. In some embodiments, in addition to intraarticular administration, metformin is systemically administered via the intravenous (IV) route more than one time. In some embodiments, in addition to intraarticular administration, metformin is administered via the intravenous (IV) route daily, every other day, once a week, twice a week, three times a week, four times a week, once every two weeks, once every three weeks, once every four weeks, once a month, once every two months, once every three months, or once every four months.

In some embodiments, in addition to intraarticular administration, metformin or a pharmaceutically acceptable salt thereof is systemically administered via the intramuscular (IM) route only once, i.e., a total of one time. In some embodiments, in addition to intraarticular administration, metformin is systemically administered via the intramuscular (IM) route more than once. In some embodiments, in addition to intraarticular administration, metformin is administered via the intramuscular (IM) route daily, every other day, once a week, twice a week, three times a week, four times a week, once every two weeks, once every three weeks, once every four weeks, once a month, once every two months, once every three months, or once every four months.

In some embodiments, metformin or a pharmaceutically acceptable salt thereof is administered via a systemic route, e.g., oral, IV, or IM, over the course of one day, two days, three days, four days, five days, six days, up to one week, up to two weeks, up to three weeks, up to four weeks, up to five weeks, up to six weeks, up to seven weeks, up to eight weeks, up to nine weeks, up to 10 weeks, up to one month, up to two months, up to three months, up to four months, up to five months, up to six months, up to seven months, up to 8 months, up to nine months, up to 10 months, up to 11 months, or up to 12 months, wherein each range is inclusive.

In some embodiments, treatment of an injured joint in a mammal according to the disclosed methods begins within about 1 hour, 3 hours, 6 hours, 12 hours, 18 hours, 24 hours, about 1 week, 2 weeks, or 3 weeks from occurrence of the injury. In preferred embodiments, treatment of an injured joint in a mammal according to disclosed methods begins less than 24 hours, less than 12 hours, or less than 6 hours from occurrence of the injury. In some embodiments, the injury involves any of a fracture, cartilage damage, acute ligament sprain, chronic ligamentous instability, tendonitis, or any combination of the same

In some embodiments, the disclosed dosing regimens are effective to treat an injured joint in a mammal, such as prevent the development of PTOA, which is evidenced by cartilage degeneration, joint pain, joint stiffness, inflammation, or a combination thereof. In some embodiments, the disclosed dosing regimens are effective to prevent the pain and/or the stiffness associated with the injured joint from worsening or reduce the pain and/or the stiffness associated with an injured joint in a mammal. In some embodiments, the disclosed dosing regimens are effective to both treat an injured joint and reduce the joint pain and/or joint stiffness associated with the injured joint in a mammal. In preferred embodiments, the mammal is human. In other preferred embodiments, the human has risk factors for joint injury, e.g., the human may be considered at high risk for joint injury. Risk factors for joint injury include, e.g., physical activity, such as sport activity (Buckwalter, J Orthop Sports Phys Ther. 2003 October 33(10):578-88) or occupational activity, e.g., related to law enforcement or military service (Roy et al., Aviat Space Environ Med. 2012 November 83(11):1060-6 and Cameron et al., J Athl Train. 2016 Novomber 51(11):952-961), and being overweight or obese (King et al., Indian J Med Res. 2013 August 138(2):185-193).

The dosing regimens described herein, including dose, dosing interval, and other aspects of the disclosed methods, may be used in a prophylactic manner, such as to prevent an injury, i.e., an injured joint, that has not yet occurred from progressing to OA, specifically PTOA. In some embodiments, the disclosed dosing regimens comprise administering metformin via the intraarticular route and/or a systemic route, such as oral, IM, or IV administration, to a mammal prior to joint injury in said mammal, for the prevention of OA, such as PTOA. In some embodiments, the disclosed dosing regimens comprise administering metformin via the intraarticular route to a mammal prior to joint injury in said mammal to prevent or reduce the pain and/or the stiffness associated with an injured joint. In some embodiments, disclosed prophylactic dosing regimens are effective to both prevent an injured joint from progressing to PTOA, such as by preventing cartilage degeneration, joint pain, joint stiffness, inflammation, or a combination thereof, and reduce the pain and/or the stiffness associated with the injured joint. In preferred embodiments, the mammal is human. In other preferred embodiments, the human has risk factors for joint injury, e.g., the human may be considered at high risk for joint injury.

### Compositions In some aspects, provided herein are compositions of metformin, such as injectable compositions comprising metformin or a pharmaceutically acceptable salt thereof. In some embodiments, the disclosed injectable compositions comprise an analog of metformin or a pharmaceutically acceptable salt thereof. In some embodiments, the disclosed injectable compositions of metformin are intended and suitable for intraarticular administration to a mammal with an injured joint. In some embodiments, disclosed injectable compositions are used to treat and/or prevent post-traumatic osteoarthritis. In some embodiments, disclosed injectable compositions are used for the manufacture of a medicament to treat and/or prevent post-traumatic osteoarthritis.

In some embodiments, the disclosed injectable compositions of metformin or a pharmaceutically acceptable salt thereof comprise metformin in an amount effective to reduce inflammation, such as mitigate generation of ROS, and preserve the function of mitochondria and viability of chondrocytes in impacted cartilage. In some embodiments, the disclosed injectable compositions of metformin comprise metformin in an amount effective to prevent tissue loss and matrix disruption. In some embodiments, the disclosed injectable compositions of metformin comprise metformin in an amount effective to prevent joint pain and/or joint stiffness. In some embodiments, the disclosed injectable compositions of metformin comprise metformin in an amount effective to prevent PTOA.

In some embodiments, the disclosed injectable compositions comprise metformin or a pharmaceutically acceptable salt thereof at a concentration of up to about 85 mM, 90 mM, 95 mM, 100 mM, 105 mM, 110 mM, 115 mM, 120 mM, 125 mM, 130 mM, 135 mM, 140 mM, 145 mM, 150 mM, 155 mM, 160 mM, 165 mM, 170 mM, 175 mM, 180 mM, 185 mM, 190 mM, 195 mM, or 200 mM. In some embodiments, disclosed injectable compositions comprise metformin or a pharmaceutically acceptable salt thereof at a concentration of up to about 100 mM, 105 mM, 110 mM, 115 mM, 120 mM, 125 mM, 130 mM, 135 mM, 140 mM, 145 mM, or 150 mM. In some embodiments, disclosed injectable compositions comprise metformin or a pharmaceutically acceptable salt thereof at a concentration of less than about 100 mM, 105 mM, 110 mM, 115 mM, 120 mM, 125 mM, 130 mM, 135 mM, 140 mM, 145 mM, or 150 mM.

In some embodiments, disclosed injectable compositions comprise metformin or a pharmaceutically acceptable salt thereof concentration of about 25 mM to 150 mM, 25 mM to 125 mM, 25 mM to 120 mM, 25 mM to 115 mM, 25 mM to 110 mM, 25 mM to 105 mM, 25 mM to 100 mM, 25 mM to 95 mM, 25 mM to 90 mM, 25 mM to 85 mM, 25 mM to 80 mM, 25 mM to 75 mM, 25 mM to 70 mM, 25 mM to 65 mM, 25 mM to 60 mM, 25 mM to 55 mM, 25 mM to 50 mM, 25 mM to 45 mM, or 25 mM to 40 mM.

In some embodiments, disclosed injectable compositions comprise metformin or a pharmaceutically acceptable salt thereof at a concentration of about 30 mM to 150 mM, 30 mM to 125 mM, 30 mM to 120 mM, 30 mM to 115 mM, 30 mM to 110 mM, 30 mM to 105 mM, 30 mM to 100 mM, 30 mM to 95 mM, 30 mM to 90 mM, 30 mM to 85 mM, 30 mM to 80 mM, 30 mM to 75 mM, 30 mM to 70 mM, 30 mM to 65 mM, 30 mM to 60 mM, 30 mM to 55 mM, 30 mM to 50 mM, 30 mM to 45 mM, or 30 mM to 40 mM.

In some embodiments, disclosed injectable compositions comprise metformin or a pharmaceutically acceptable salt thereof at a concentration of about 35 mM to 150 mM, 35 mM to 125 mM, 35 mM to 120 mM, 35 mM to 115 mM, 35 mM to 110 mM, 35 mM to 105 mM, 35 mM to 100 mM, 35 mM to 95 mM, 35 mM to 90 mM, 35 mM to 85 mM, 35 mM to 80 mM, 35 mM to 75 mM, 35 mM to 70 mM, 35 mM to 65 mM, 35 mM to 60 mM, 35 mM to 55 mM, 35 mM to 50 mM, 35 mM to 45 mM, or 35 mM to 40 mM.

In some embodiments, disclosed injectable compositions comprise metformin or a pharmaceutically acceptable salt thereof at a concentration of about 40 mM to 150 mM, 40 mM to 125 mM, 40 mM to 120 mM, 40 mM to 115 mM, 40 mM to 110 mM, 40 mM to 105 mM, 40 mM to 100 mM, 40 mM to 95 mM, 40 mM to 90 mM, 40 mM to 85 mM, 40 mM to 80 mM, 40 mM to 75 mM, 40 mM to 70 mM, 40 mM to 65 mM, 40 mM to 60 mM, 40 mM to 55 mM, 40 mM to 50 mM, or 40 mM to 45 mM.

In some embodiments, disclosed injectable compositions comprise metformin or a pharmaceutically acceptable salt thereof at a concentration of about 45 mM to 150 mM, 45 mM to 125 mM, 45 mM to 120 mM, 45 mM to 115 mM, 45 mM to 110 mM, 45 mM to 105 mM, 45 mM to 100 mM, 45 mM to 95 mM, 45 mM to 90 mM, 45 mM to 85 mM, 45 mM to 80 mM, 45 mM to 75 mM, 45 mM to 70 mM, 45 mM to 65 mM, 45 mM to 60 mM, 45 mM to 55 mM, or 45 mM to 50 mM.

In some embodiments, disclosed injectable compositions comprise metformin or a pharmaceutically acceptable salt thereof concentration of about 50 mM to 150 mM, 50 mM to 125 mM, 50 mM to 120 mM, 50 mM to 115 mM, 50 mM to 110 mM, 50 mM to 105 mM, 50 mM to 100 mM, 50 mM to 95 mM, 50 mM to 90 mM, 50 mM to 85 mM, 50 mM to 80 mM, 50 mM to 75 mM, 50 mM to 70 mM, 50 mM to 65 mM, 50 mM to 60 mM, or 50 mM to 55 mM.

In some embodiments, disclosed injectable compositions comprise metformin or a pharmaceutically acceptable salt thereof concentration of about 55 mM to 150 mM, 55 mM to 125 mM, 55 mM to 120 mM, 55 mM to 115 mM, 55 mM to 110 mM, 55 mM to 105 mM, 55 mM to 100 mM, 55 mM to 95 mM, 55 mM to 90 mM, 55 mM to 85 mM, 55 mM to 80 mM, 55 mM to 75 mM, 55 mM to 70 mM, 55 mM to 65 mM, or 55 mM to 60 mM.

In some embodiments, disclosed injectable compositions comprise a pharmaceutically acceptable salt of metformin, such as metformin hydrochloride. In some embodiments, the pharmaceutically acceptable salt of metformin is metformin benzoate, metformin ethanesulfonate, metformin (2:1) fumarate, metformin glycinate, metformin glycolate, metformin hydrobromide, metformin hydrochloride, metformin maleate, metformin phosphate, metformin salicylate, metformin (2:1) succinate, and metformin sulfate. Such metformin salts are described in BE568513A, U.S. Pat. No. 8,703,183B2, U.S. Pat. No. 6,031,004A, and U.S. Pat. No. 3,174,901A. Additional metformin formulations, which may be incorporated with disclosed compositions, include fatty acid complexes, e.g., as described in US20050158374A1, lipophilic acid salts, e.g., as described in US20030220301A1, and controlled release formulations, such as described in US20060034922A1 and WO 99/47128. In preferred embodiments, disclosed compositions comprise metformin hydrochloride.

Additional pharmaceutically acceptable salts of metformin which may be included in disclosed injectable compositions include the (4-chlorophenoxy) isobutyrate salt (FR2275199A1), acetylsalicylate acid salt (U.S. Pat. No. 3,957,853A), chlorophenoxyacetic acid salt (U.S. Pat. No. 4,835,184A), clofribrate salt (U.S. Pat. No. 4,080,472A), dichloroacetic acid salt (U.S. Pat. No. 4,028,402A), nicotinic acid salt (DE2357864A1), orotate salt (FR2264539A1), pamoate salt (FR2037002A1 and FR2320735A1).

In some embodiments, disclosed injectable compositions comprise any one or more of atelocollagen, tripeptide collagen, and type-I polymerized collagen, and type-II collagen. In some embodiments, disclosed injectable compositions do not include compounds known and used to induce osteoarthritis, such as mice with bovine or chicken type II collagen (CII) emulsified in complete Freund's adjuvant (CFA).

In some embodiments, the injectable compositions of metformin or a pharmaceutically acceptable salt thereof further comprise a hydrogel. In some embodiments, a disclosed solution of metformin is loaded into a hydrogel prior to intraarticular administration. In some embodiments, the hydrogel is an injectable composite hydrogel comprising a semi-synthetic polysaccharide. In some embodiments, the hydrogel is an injectable composite hydrogel comprising a synthetic polysaccharide. In some embodiments, the hydrogel is an injectable composite hydrogel comprising a synthetic polymer. In some embodiments, the hydrogel has reverse-thermal properties, wherein the composition becomes firm once injected, thereby preventing leakage from the injection site.

In some embodiments, the hydrogel is an injectable composite hydrogel comprising a polysaccharide. In some embodiments, the polysaccharide is a natural polysaccharide. In some embodiments, the natural polysaccharide comprises any one or more of hyaluronic acid, hydroxypropylcellulose, karya gum (KG), guar gum (GUG), or gellan gum (GEG). In some embodiments, the polysaccharide is present in the hydrogel at about 0.1% (wt/vol) to about 10%, 0.1% (wt/vol) to about 5.0%, 0.1% (wt/vol) to about 1.0% (wt/vol), including values in between and wherein each range is inclusive.

In preferred embodiments, the disclosed injectable compositions comprise hyaluronic acid. In some embodiments, the hyaluronic acid is linear or cross-linked. In some embodiments, the hydrogel comprising hyaluronic acid comprises about 0.1% wt/vol to about 10 % wt/vol, 0.1% wt/vol to about 5 % wt/vol, or 0.1% wt/vol to about 1 % wt/vol hyaluronic acid. In some embodiments, the hydrogel comprising hyaluronic acid comprises about 1% wt/vol, 2% wt/vol, 3% wt/vol, 4% wt/vol, 5% wt/vol, 6% wt/vol, 7% wt/vol, 8% wt/vol, 9% wt/vol, or 10% wt/vol hyaluronic acid. In some embodiments, the polysaccharide comprises hyaluronic acid of about or greater than 1.5 M Dalton. In one embodiment, the MW is about 1,600,000 to 3,200,000, or about 1,900,000 to 3,900,000.

In some embodiments, the hydrogel comprising hyaluronic acid further comprises at least one excipient. In some embodiments, the at least one excipient is any one or more of sodium chloride, dibasic sodium phosphate dodecahydrate, and sodium dihydrogen phosphate dihydrate.

In other preferred embodiments, the disclosed injectable compositions comprise a salt form of hyaluronic acid, e.g., sodium hyaluronate and/or potassium hyaluronate. In some embodiments, the hyaluronate is linear or cross-linked. In some embodiments, the hydrogel comprising hyaluronate comprises about 0.1% wt/vol to about 10 % wt/vol, 0.1% wt/vol to about 5 % wt/vol, or 0.1% wt/vol to about 1 % wt/vol hyaluronate. In some embodiments, the hydrogel comprising hyaluronate comprises about 1% wt/vol, 2% wt/vol, 3% wt/vol, 4% wt/vol, 5% wt/vol, 6% wt/vol, 7% wt/vol, 8% wt/vol, 9% wt/vol, or 10% wt/vol hyaluronate. In some embodiments, the hyaluronate is sodium hyaluronate (hyaluronan).

In some embodiments, the hydrogel comprising hyaluronate further comprises at least one excipient. In some embodiments, the at least one excipient is any one or more of sodium chloride, dibasic sodium phosphate dodecahydrate, and sodium dihydrogen phosphate dihydrate.

Known injectable hyaluronic acid compositions may also be modified by the addition of metformin to practice the methods disclosed here. Such compositions, which include injectable compositions of hyaluronic acid and derivatives thereof, such as for intraarticular injection, and including hydrogels comprising the same, are described in, e.g., US20040053885A1, EP3334411B1, U.S. Pat. No. 8,889,652B2, WO2022225476A1, and CN114504547A, which are incorporated by reference herein.

In some embodiments, the disclosed injectable compositions comprise a composite reverse-temperature sensitive hydrogel further comprising a biopolymer, such as a polysaccharide, and/or a synthetic polymer. In one embodiment, the composition is a reverse temperature-sensitive hydrogel, one that is non-viscous at “low” temperature, e.g., at or below room temperature, e.g., about 70° F. or less. The low initial viscosity allows the hydrogel to coat all the cartilage surfaces through the joint before it sets, i.e., the viscosity increases at temperatures above room temperature, e.g., about 80° F. or greater including human body temperature such as about 98°0 F., which provides for superior retention in the joint and substantially improves the bioavailability of the compound dissolved in the gel. Reverse temperature-sensitive hydrogels, which have initial viscosities of about 100 to about 160 or about 80 to about 200, e.g., about 120 to about 140, Pascal Seconds, may be administered using a 22 to 24 gauge needle, e.g., a 22 gauge needle. In contrast, non-reverse temperature-sensitive hydrogels require large bore needles and do not evenly distribute in the joint due to their high initial viscosity.

In some embodiments, a disclosed injectable composition comprises a synthetic polymer. In some embodiments, the synthetic polymer comprises any of carboxyvinyl-polymers, e.g., Carbopol 934, cellulose derivatives, e.g., methylcellulose, cellulose acetate and hydroxypropyl cellulose, polyacrylamide, polyvinyl pyrrolidone, polyvinyl alcohols, N-isopropyl acrylamide polymer, ethyl hydroxyethyl cellulose, poly(ethylene oxide-b-propylene oxide-b-ethylene oxide), poloxamers, such as F127, synthetic block copolymers which consist of hydrophilic poly(ethylene oxide) (PEO) and hydrophobic poly(propylene oxide), poly(ethylene glycol)/poly(D, L-lactic acid-co-glycolic acid) block copolymers, synthetic polysaccharides, alginate, polyphosphazenes, polyacrylates, or polyethylene oxide-polypropylene glycol block copolymers, hydroxyapatite, poly(epsilon-caprolactone)-poly(ethylene glycol) copolymers, polyacryloyl hydroxyethyl) starch, or agarose or the preceding in any combination. In some embodiments, the hydrogel includes about 1% wt/vol to about 25% wt/vol of a synthetic polymer.

In some embodiments, the hydrogel includes collagen, e.g., hydroxylated collagen, fibrin, polylactic-polyglycolic acid, or a polyanhydride. In preferred embodiments, a disclosed injectable composition does not comprise collagen. Other examples include, without limitation, any biocompatible polymer, whether hydrophilic, hydrophobic, or amphiphilic, such as ethylene vinyl acetate copolymer (EVA), polymethyl methacrylate, polyamides, polycarbonates, polyesters, polyethylene, polypropylenes, polystyrenes, polyvinyl chloride, Polytetrafluoroethylene, N-isopropylacrylamide Copolymers, Polyfethylene oxide)/poly(propyiene oxide) block copolymers, poly(ethylene glycol)/poly(D, L-lactide-co-glycolide) block copolymers, polyglycolide, polylactides (PLLA or PDLA), poly(caprolactone) (PCL), or poly(dioxanone) (PPS).

In some embodiments, a disclosed injectable composition comprises one or more biocompatible polymers. In some embodiments, the biocompatible polymer is any of polylactic-co-glycolic acid, poly(-caprolactone) (PCL), polylactic acid, poly(3-hydroxybutyrate-co-3-hydroxyvalerate) (PHBV), chitosan, cellulose, or any combination of the preceding.

In some embodiments, a disclosed injectable composition comprises one or more natural polymers. In some embodiments, the natural polymer is alginate, agarose, starch, fibrin, collagen, gelatin, chitin, glycosaminoglycans, e.g., hyaluronic acid, dermatan sulfate and chondroitin sulfate, and microbial polyesters, e.g., polyhydroxyalkanoates, such as hydroxyvalerate and hydroxybutyrate copolymers, and synthetic polymers, e.g., poly(orthoesters) and polyanhydrides, and including homo and copolymers of glycolide and lactides (e.g., poly(L-lactide, poly(L-lactide-co-D,L-lactide), poly(L-lactide-co-glycolide, polyglycolide, poly(D,L-lactide), poly(D,L-lactide-co-glycolide), polylactic acid co-lysine), polycaprolactone, silk, or the preceding in any combination. In some embodiments, the natural polymer is biocompatible.

In some embodiments, a disclosed injectable composition comprises at least one additional compound that is chondroprotective, prevents cartilage degradation, or otherwise is used to treat OA, such as PTOA. In some embodiments, the additional compound inhibits mitochondrial dysfunction or chondrocyte energy dysfunction. In some embodiments, the additional compound scavenges mitochondrial oxidants, prevents their formation, or stimulates glycolytic ATP production. Such compounds are known to one of skill in the art and are described in, e.g., U.S. Pat. No. 7,067,144B2, U.S. Pat. No. 10,300,073B2, U.S. Pat. No. 9,498,471B2, US20220071963A1, US20190015384A1, US20180064712A1, US20100215731A1, and US20210222177A1, which are incorporated by reference herein.

In some embodiments, the components of the composition may be administered by infusion or injection. Solutions may be prepared in water, optionally mixed with a nontoxic surfactant. Dispersions may also be prepared in glycerol, liquid polyethylene glycols, triacetin, and mixtures thereof and in oils. Under ordinary conditions of storage and use, these preparations contain a preservative to prevent the growth of microorganisms. The pharmaceutical dosage forms suitable for injection or infusion may include sterile aqueous solutions or dispersions or sterile powders comprising the active ingredient which are adapted for the extemporaneous preparation of sterile injectable or infusible solutions or dispersions. In all cases, the ultimate dosage form should be sterile, fluid, and stable under the conditions of manufacture and storage. The liquid carrier or vehicle may be a solvent or liquid dispersion medium comprising, for example, water, ethanol, a polyol, for example, glycerol, propylene glycol, liquid polyethylene glycols, and the like, vegetable oils, nontoxic glyceryl esters, and suitable mixtures thereof. The prevention of the action of microorganisms can be brought about by various antibacterial and antifungal agents, for example, parabens, chlorobutanol, phenol, sorbic acid, thimerosal, and the like. In many cases, it may be preferable to include isotonic agents, for example, sugars, buffers, or sodium chloride.

Sterile injectable solutions may be prepared by incorporating the active agent in the required amount in the appropriate solvent with various other ingredients, as required, optionally followed by filter sterilization. In the case of sterile powders for the preparation of sterile injectable solutions, the methods of preparation include vacuum drying and freeze-drying techniques, which yield a powder of the active ingredient plus any additional desired ingredient present in the previously sterile-filtered solutions.

### EXAMPLES

### Example 1: High Dose Metformin is Necessary to Reduce Apoptosis, Suppress Oxidative Stress, Preserve Chondrocyte Viability, and Rescue Mitochondrial Dysfunction in Impacted Chondrocytes

Bovine cartilage explants were impacted with sublethal force to evaluate the injury response in viable chondrocytes. The effects of metformin hydrochloride at various concentrations were then evaluated in the contexts of apoptosis, oxidative stress, chondrocyte viability, and mitochondrial dysfunction.

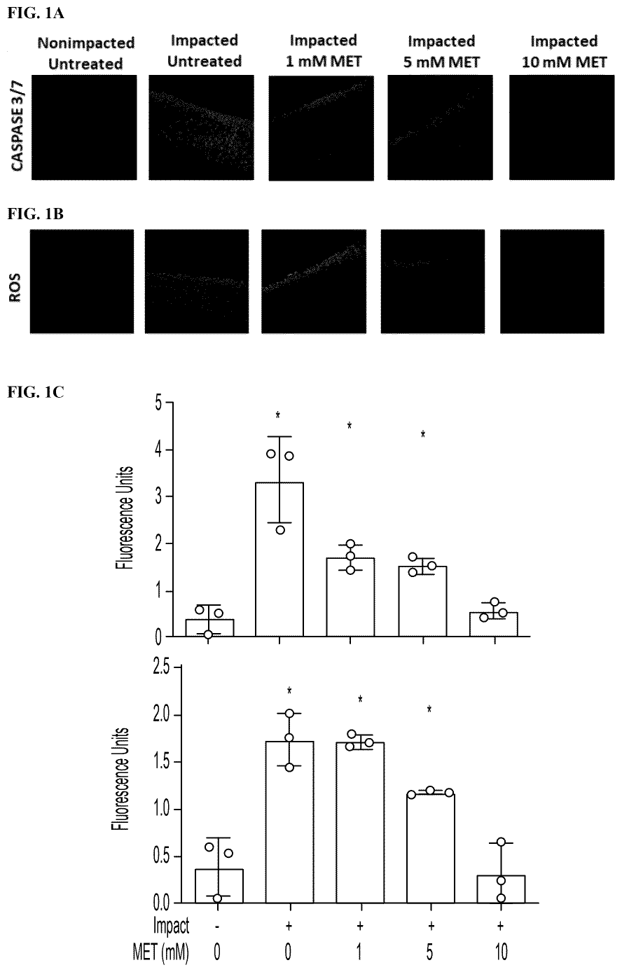

Apoptosis and Oxidative Stress: Cartilage was removed from subchondral bone and stained for apoptosis via activated caspase 3/7signalling (CellEvent, Invitrogen) or ROS (CellRox, Invitrogen) 24 hr post-impact, as shown in FIG. 1A and FIG. 1B, respectively. Quantification of stain intensity in confocal images revealed a significant increase in apoptosis with impact, and metformin treatment resulted in a dose-dependent decrease in the same (FIG. 1C-t op). However, a concentration of 1 mM metformin only partially reduced apoptotic signalling, whereas a higher concentration of 10 mM completely suppressed apoptosis. Images and quantification of oxidative stress (FIG. 1C-b ottom) showed similar results, with full suppression of oxidative stress resulting from 10 mM metformin.

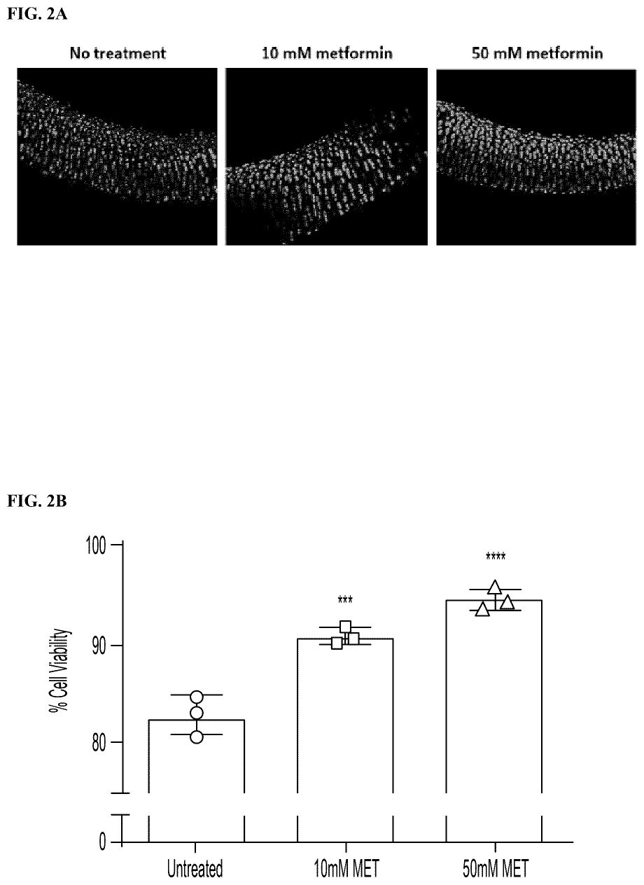

Chondrocyte Viability: As 10 mM metformin hydrochloride was required to suppress markers of apoptosis and oxidative stress following a sublethal impact overload to cartilage, this concentration was chosen as a baseline assess preservation of chondrocyte viability following a more severe mechanical overload. Bovine cartilage was cultured with metformin following an impact from a drop tower device. Confocal images of live/dead staining (Invitrogen) 24 hr post-impact revealed that while 10 mM metformin improved cell viability, 50 mM was necessary to completely rescue impacted chondrocytes (FIG. 2A and FIG. 2B).

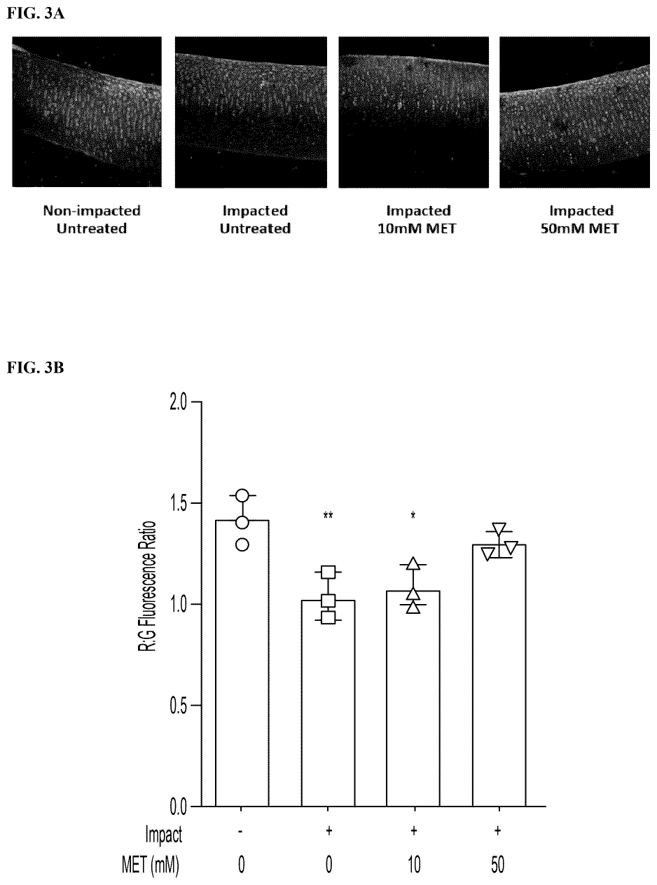

Mitochondrial Function: The integrity of the mitochondrial membrane was assessed using confocal microscopy of fluorescent probes. FIG. 3A shows metformin-treated impacted bovine explants incubated with tetramethylrhodamine methyl ester perchlorate (TMRM; Molecular Probes) and MitoTracker Green (Molecular Probes). TMRM is a polarity-sensitive mitochondrial probe, and its red fluorescence indicates active transport of the dye across a polarized, functional mitochondrial membrane. MitoTracker Green is a polarity-insensitive stain which stains all mitochondria with green fluorescence.

The ratio of the fluorescent intensity of red and green stains for each explant was determined with ImageJ to represent the relative mitochondrial membrane polarity 24 hours post-impact. Mitochondrial polarity was significantly decreased in impacted explants and was rescued with 50 mM metformin (FIG. 3B), indicating that metformin rescues mitochondrial dysfunction in impacted chondrocytes in a dose-dependent manner. The concentration of metformin necessary to prevent cell death and rescue mitochondrial dysfunction (50 mM) necessitates delivery by intraarticular injection, as oral administration at clinical levels would deliver an insufficient amount of drug to the joint.

### Example 2: Activation of AMPK and SIRT1 Preserves the Viability of Impacted Chondrocytes

The pharmacology of metformin is complex and includes activation of AMPK and SIRT1. The effects of activating AMPK or SIRT1 in injured chondrocytes were assessed to determine whether the molecular targets mediate prevention of cartilage damage.

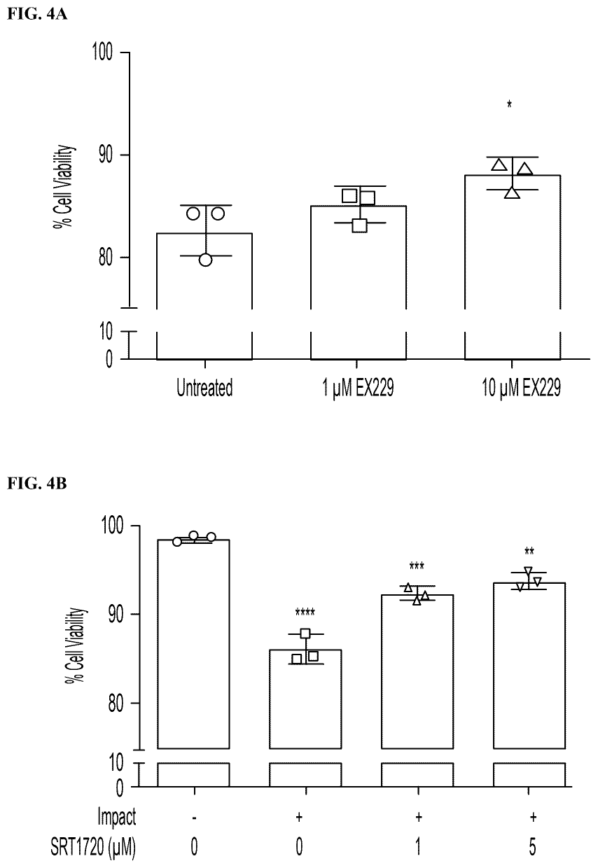

As a preliminary assessment, live/dead staining was evaluated in impacted bovine cartilage treated with specific pharmacological activators of AMPK and SIRT1 or left untreated with appropriate vehicle (DMSO). Live/dead staining indicated that AMPK activator EX229 (FIG. 4A) and SIRT1 activator SRT1720 (FIG. 4B) improved cell viability in impacted chondrocytes in a dose-dependent manner. For example, FIG. 4A shows that AMPK activation rescues the AMD-induced decrease in chondrocyte viability, and FIG. 4B indicates that SIRT1 activation rescues chondrocyte viability following AMD. However, neither of these activators completely prevented impact-induced chondrocyte death. Additionally, the low solubility of the compounds in aqueous solutions may undermine drug delivery and clinical translation.

These results indicate that activating AMPK and SIRT1 may be an effective strategy to treat impacted chondrocytes and prevent the progression of PTOA. In addition to inhibiting Complex I, which has been shown to reverse markers of cartilage injury, metformin also activates AMPK and SIRT1. Accordingly, metformin may treat the impact-induced cartilage injury response through multiple molecular targets, including by activating AMPK and SIRT1.

### Example 3: Determining the Therapeutic Ceiling of Metformin in Impacted Bovine Cartilage

To determine whether higher concentrations of metformin would continue to exhibit a therapeutic dose-dependent effect on injured chondrocytes, impacted bovine cartilage was treated with up to 125 mM metformin hydrochloride. One half of the explant was assessed for cell viability via live/dead staining, and the other half was stained with MitoTracker Green and TMRM to evaluate mitochondrial health.

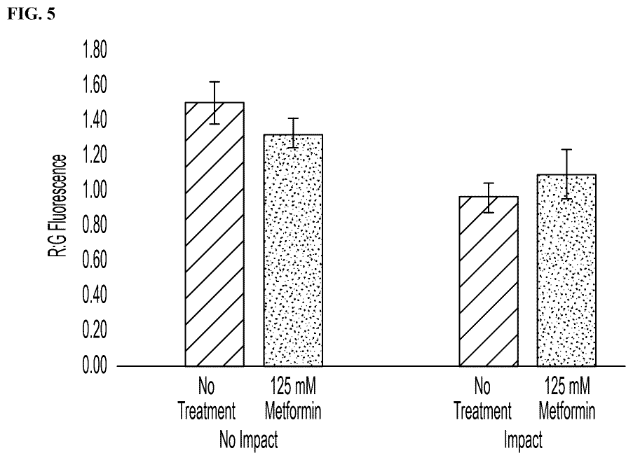

Surprisingly, as shown in FIG. 5, the mitochondrial function of non-impacted chondrocytes slightly declined following treatment with 125 mM metformin hydrochloride. Additionally, the mitochondrial function of impacted chondrocytes treated with 125 mM metformin hydrochloride was not significantly different from that of untreated impacted chondrocytes (FIG. 5). Cell viability showed similar trends (data not shown). Together, these data indicate a therapeutic ceiling for metformin in the context of treating injured chondrocytes.

### Example 4: Intraarticular Metformin Prevents the Progression of Trauma-Induced Osteoarthritis in Rabbits

A cartilage impact injury in a rabbit was developed as a preclinical model of PTOA to investigate mechanisms of disease progression and test novel therapeutics that prevent or delay the onset of joint disease. A rabbit model was chosen because the joint must be mechanically stabilized with hardware during the impact, and the rabbit is the smallest animal for which this procedure is feasible. This model involves a single direct impact to the medial femoral condyle with a metal impactor head, resulting in immediate AMD to the cartilage tissue and PTOA within 8 to 16 weeks.

Validation of Animal Model: A posterior approach was used to expose the posterior medial femoral condyle in the knees in anesthetized Zealand White rabbits weighing 3.0-4.2 kg (FIG. 2). A 1/16″ diameter k-wire was inserted through both condyles in the medial-lateral direction and secured to a platform. A drop tower with a 3 mm diameter impactor head mounted on the carriage had an impacting surface with radii of curvature approximated to the femoral condyle. With the impact head positioned over the medial femoral condyle, the carriage was dropped 7 cm. Rabbits were euthanized at 16-weeks post-impact, and knees were harvested for sectioning and staining with Safranin-O Fast Green for OARSI scoring (n=8).

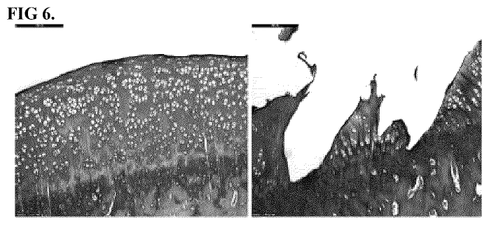

FIG. 6 shows representative safranin-O staining of non-impacted (left) and impacted (right) medial condyles from rabbit knees. OARSI scores were 1.6±0.53 in non-impacted tissue and 5.8±2.3 in impacted condyles. For each specimen, the most progressive stage of OA was within the zone of impact. Accordingly, the rabbit model of cartilage impact injury mimics high-energy joint trauma that results in the tissue loss and matrix disruption characteristic of PTOA.

Metformin Studies: Right medial condyles of rabbits were impacted and either treated with 200 μl hydrogel or a hydrogel containing 50 mM metformin hydrochloride. Hydrogel alone or loaded with 50 mM metformin was intraarticularly administered after the impact, while animals were still under anesthesia from the impact surgery.

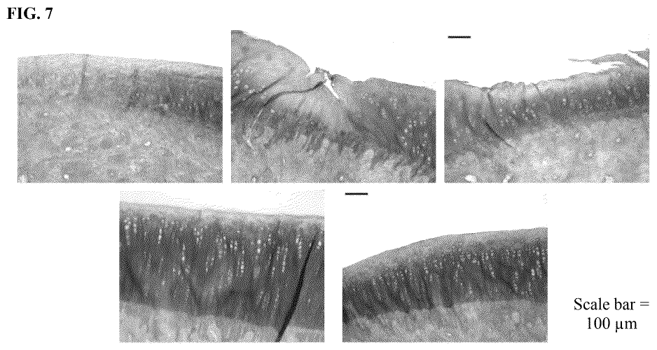

Tissue was harvested 12 weeks post-impact, processed for histological sectioning, and stained with Safranin O. As depicted in FIG. 7, specimens treated solely with the hydrogel experienced tissue loss and matrix disruption in the zone of impact, with OARSI scores of 7.0±2.6. Histological sections from tissue treated with 50 mM metformin-loaded hydrogel showed minimal damage, with OARSI scores of 0 and 1, indicating a marked improvement in cartilage health and prevention of PTOA.

### Example 5: Toxicity Testing of Metformin in Human Chondrocytes

Healthy human cartilage samples from will be collected from femoral heads of hip fracture patients. At least four cartilage explants 5 mm in diameter will be isolated from each donor. Tissue will be cultured in 0, 10 mM, 50 mM and 125 mM of a pharmaceutically acceptable salt of metformin for 3 days. Cell viability and mitochondrial function, among other measures described herein, will be evaluated to assess the safety and toxicity of the various metformin concentrations. Concentrations of a pharmaceutically acceptable salt of metformin up to 50 mM are not expected to exhibit cytotoxic effects.

### Example 6: Determining the Effects of Local and Systemic Administration of Metformin Immediately Following Injury and Two Weeks Post-Injury in Rabbits

Rabbit Impact Model: A single controlled blunt impact injury will be delivered to the stabilized knees of 7-8-month-old New Zealand White rabbits. After animals undergo anesthesia as above, the leg will be shaved and prepared with sterile technique and the animals will be positioned prone in a custom-fabricated surgical cradle. The femur will be stabilized with a percutaneous 1.6 mm Kirschner (k) transfixion wire inserted through the lateral and medial condyles and secured in adjustable clamps on the cradle with the knee in maximal extension. A posterior arthrotomy will be made through a longitudinal 3-4 cm incision centered over the medial popliteal fossa. The posteromedial joint capsule will be exposed through the intermuscular plane between the semi-membranous and medial gastrocnemius. The gastrocnemius tendon will be retracted laterally, with the femoral insertion left intact. The joint capsule will then be incised longitudinally exposing the posterior weight bearing surface of the medial femoral condyle. A drop tower apparatus with a sterile stainless steel impactor tip (circular 3 mm diameter with concave impacting surface) will be brought into the surgical field. The impactor tip will be brought into contact with the posterior medial femoral condyle. Once the position is optimized, the drop tower will be secured to the surgical cradle. The impactor will be raised to 7 cm above the articular surface and a subfracture impact will be delivered with expected impact parameters consistent with the methods described herein. The joint will be irrigated with sterile saline and the arthrotomy and skin will be closed with polysorb and monofilament sutures, respectively.

Intraarticular Injection of Metformin: For intraarticular delivery, 50 mM metformin hydrochloride (Sigma) will be loaded into 0.2% crosslinked hyaluronate hydrogel further comprising sodium chloride, dibasic sodium phosphate dodecahydrate, sodium dihydrogen phosphate dihydrate, and water for injection (GelOne®, Zimmer Biomet, Warsaw IN) to aid retention in the joint. Concentrated metformin will be made in sterile PBS, filtered, and aliquoted. Separate aliquots of the hyaluronate hydrogel at the appropriate concentration will be prepared without metformin for the untreated group. Aliquots from each solution will be tested for endotoxins (ToxinSensor Endotoxin Detection System, Genescript, Piscataway, NJ). After the incisions are closed, 200 μL of the solution will be injected into the knees for immediate treatments. Alternately, rabbits will receive delayed intraarticular injection at 2 weeks post-trauma.

Systemic Administration of Metformin: Rabbits limit their intake of food and water immediately after surgery. Accordingly, systemic administration of metformin on the day of surgery will be achieved by intravenous injection of 52 mg/kg metformin in saline via the ear vein, while the animal is still under anesthesia. On the following days, metformin (Sigma) will be added to the rabbits' drinking water on a daily basis. Rabbits' water consumption and weight will be measured, and weekly adjustments will be made to individual water compositions to deliver 52 mg/kg metformin per day. Based on the surface area of the rabbit body, this dose is the human equivalent dose of 1000 mg per day. This dose is also equivalent to oral and intraperitoneal metformin doses in mouse models that have been effective at combatting other types of OA, using the same conversion based on body surface area. This protocol will be followed for treatment beginning the day after impact injury and continuing for the study duration.

Joint Health Outcomes: Pain behaviour scores will be recorded by blinded personnel at least once per day for 2 weeks following surgery and/or until scores return to baseline levels. At 16 weeks, whole impacted or contralateral knee joints from rabbits will be fixed in neutral buffered 10% formalin after harvesting. High-resolution μCT image analyses of the femorotibial joint subchondral bone will be performed to measure trabecular bone volume (% BV/TV), trabecular number (Tb.N), thickness (Tb.Th) and separation (Tb.Sp). Ten consecutive images from the medial plateau in the coronal plane will be utilized to measure the subchondral bone plate thickness (SBP. Th-cortical bone). The same whole articulated knee joints will be decalcified, paraffin-embedded, and 5 μm thick serial sections will be histologically stained with hematoxylin-eosin (H&E) to measure cartilage and bone parameters, and safranin O fast green (SO) to measure cartilage proteoglycan content.

Histological assessment of OA will be performed using the OARSI scoring system by three blinded investigators. The OARSI score is based on six grades that reflect depth of the lesions and four stages reflecting the extent of degradation over the joint surface, with a total maximum score of 24. Synovitis will also be evaluated using the OARSI system on H&E-stained sections. Macrophage infiltration of the synovial tissues will be evaluated via IHC against F4/80. Type 2 collagen and aggrecan content of cartilage will be assessed via IHC. Analysis of apoptosis will be performed via terminal deoxynucleotidyl transferase dUTP nick end labeling (TUNEL) staining.

Safety and Toxicity Outcomes: Body weights of rabbits will be recorded on a weekly basis from the start and blood will be collected monthly as well as at the beginning and end of the study for serum analysis of indicators of hypoglycemia and renal and hepatic toxicity. Untreated cohorts will serve as controls. One half of each explant will be stained with Live/Dead reagents and fluorescence will be imaged with confocal microscopy. The other half will be homogenized and energy production via ATP synthesis will be evaluated with a commercial Luciferin-Luciferase Assay Kit. Cell lysates will be analyzed using a luminescence reader and normalized to DNA content (Picogreen, Invitrogen). Paired statistics will be used to analyze the data.

The articular cartilage of the untreated cohort is expected to show significant arthritic changes, including matrix damage, chondrocyte death, and proteoglycan loss, which are typical of PTOA. Immediate intraarticular injection of metformin may protect chondrocytes against cell death, mitochondrial dysfunction, oxidative stress, and apoptotic signalling associated with AMD, thereby preventing the progression of cartilage injury to PTOA. Further, the combination of immediate intraarticular injection and systemic delivery may be particularly effective, as the sustained oral treatment may rescue any chronic aberrant cellular behavior due to RME.

Delayed intraarticular injection or systemic delivery of metformin may improve cartilage health but may not be fully effective, as cell viability will likely be compromised by unmitigated AMD. However, the combination of delayed intraarticular injection and systemic delivery may provide a more robust treatment.

As metformin has shown promise in pain management pain behavior, scores in treated animals may decrease to baseline more rapidly than in untreated controls. Significant changes in blood serum markers in treated rabbits are not expected.

### Example 7: Determining the Effects of Local and Systemic Administration of Metformin Immediately Following Injury and One-Week Post-Injury in Pigs

Yucatan minipigs will be subjected to the pilon-equivalent hock joint injury, as has been reported previously by Goetz et al., Osteoarthritis Cartilage. 2015 Oct; 23(10):1797-805. The fracture will be immediately reduced anatomically and stabilized with internal fixation, minimizing changes in the RME. After animals undergo anesthesia as above, animals will be placed in the lateral position. An antero-medial approach will be used to expose the anterior tibia, and a posterior-medial approach will be used to expose the distal talus. Peri-articular soft tissues such as the ligaments and joint capsule will be preserved. Under fluoroscopic visualization, an impact plate will be attached to the talus with three tapered conical fixator pins. A stress-riser cut will be made in the distal tibia with a saw across its full width, stopping 1-2 mm from the subchondral bone, to generate stress concentrations during impact and a predictable fracture pattern. A plate that articulates with a ball joint will be secured to the distal tibia 3 mm proximal to the stress-riser cut. The plate on the tibia will be anchored to a custom leg holder that includes housing for external fixation pins. A pendulum will impart the 40 J impact injury to the impact plate, driving the talus into the anterior tibia and fracturing the distal tibia at the stress-riser cut. The fracture will be reduced with internal from with a veterinary-grade 2.7 mm TPLO plate held in place with bicortical screws proximal and distal to the fracture. Animals will be casted for 1 week after fracture and allowed to bear weight as tolerated.

Intraarticular Injection of Metformin: For intraarticular delivery, 50 mM metformin hydrochloride (Sigma) will be loaded into the same GelOne® HA hydrogel, as described in Example 6. Separate hydrogels will be prepared without metformin for the untreated group. Aliquots from each solution will be tested for endotoxins (ToxinSensor Endotoxin Detection System, Genescript, Piscataway, NJ). After internal fixation is in place, 500 μL of hydrogel will be injected into the knees for immediate treatments. A delayed treatment group will receive intraarticular metformin 1 week following injury.

Systemic Metformin Treatment: Metformin in pill form (Covetrus) will be given to the minipigs hidden in food treats. Minipig weights will be measured weekly, and weekly adjustments will be made to deliver metformin at approximately 18 mg/kg body weight per day. Considering minipig body surface area, 18 mg/kg is the human equivalent dose of 1000 mg per day. This dose is also equivalent to oral metformin doses in mouse models that have been effective at combatting other types of OA using the same conversion based on body surface area. This protocol will be followed for treatment beginning the day after hock injury and continuing for the duration of the study.

Joint Health Outcomes: X-rays will be taken at the time of injury and at 2, 4, and 6 months post-injury. X-rays will be scored on the Kellgren-Lawrence scale by three blinded investigators. Pain behavior scores will be recorded by blinded personnel at least once per day for 2 weeks following surgery and/or until levels return to baseline. Additionally, mobility of the animals will be measured by blinded personnel. Minipigs will be released into an area that contains a food treat at a set distance away. The time it takes the animal to reach the treat will be recorded and will be compared between groups.

At 6 months, impacted or contralateral knee joints from pigs will be fixed in neutral buffered 10% formalin after harvesting. Joints will be decalcified, paraffin-embedded, and 5 μm thick serial sections will be histologically stained with hematoxylin-eosin (H&E) to measure cartilage and bone parameters, and safranin O fast green (SO) to measure cartilage proteoglycan content. Histological assessment of OA will be performed using the OARSI scoring system by three blinded investigators. The OARSI score is based on six grades that reflect depth of the lesions and four stages reflecting the extent of degradation over the joint surface (total maximum score of 24). Type 2 collagen and aggrecan content of cartilage will be assessed via immunohistochemistry. Analysis of apoptosis will be performed via TUNEL staining.

Safety and Toxicity Outcomes: Body weights of minipigs will be recorded on a weekly basis from the beginning of the experiment. Blood will be collected at 2, 4, and 6 months post-impact, as well as at the beginning and end of the study, for analysis of serum indicators of hypoglycemia and renal and hepatic toxicity. Untreated cohorts will serve as controls.

The articular cartilage of the untreated cohort is expected to exhibit significant arthritic changes, including matrix damage, chondrocyte death, and proteoglycan loss typical of PTOA. Further, we expect outcomes with metformin treatments to be consistent with those of the rabbit studies described in Examples 4 and 6, as both are models of PTOA arising from AMD. Immediate intraarticular injection of metformin may prevent the progression of cartilage injury to PTOA. Delayed intraarticular injection of metformin may also interfere with the pathogenesis of PTOA but to a lesser extent than immediate intraarticular administration of metformin. However, the combination of intraarticular and systemic metformin may provide a more effective treatment. As metformin has shown promise in pain management, pain behaviour scores in treated animals may decrease more rapidly than in untreated controls. Likewise, mobility may be better in animals treated with metformin. Significant changes in blood serum markers related to hypoglycemia and renal and hepatic toxicity in treated minipigs are not expected.

### EQUIVALENTS AND SCOPE

Those skilled in the art will recognize or be able to ascertain using no more than routine experimentation many equivalents to the specific embodiments described herein. The scope of the present invention is not intended to be limited to the above, but rather is as set forth in the appended claims.

In the claims articles such as “a,” “an,” and “the” may mean one or more than one unless indicated to the contrary or otherwise evident from the context. Claims or descriptions that include “or” between one or more members of a group are considered satisfied if one, more than one, or all the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all the group members are present in, employed in, or otherwise relevant to a given product or process.

Furthermore, it is to be understood that the invention encompasses all variations, combinations, and permutations in which one or more limitations, elements, clauses, and descriptive terms, from one or more of the listed claims is introduced into another claim. For example, any claim that is dependent on another claim can be modified to include one or more limitations found in any other claim that is dependent on the same base claim.

Where elements are presented as lists, e.g., in Markush group format, it is to be understood that each subgroup of the elements is also disclosed, and any element(s) can be removed from the group. It should be understood that, in general, where the invention, or aspects of the invention is/are referred to as comprising particular elements, features, etc., certain embodiments of the invention or aspects of the invention consist, or consist essentially of, such elements, features, etc. For purposes of simplicity, those embodiments have not been specifically set forth in haec verba herein. It is also noted that the term “comprising” is intended to be open and permits the inclusion of additional elements or steps.

Where ranges are given, endpoints are included. Furthermore, it is to be understood that unless otherwise indicated or otherwise evident from the context and understanding of one of ordinary skill in the art, values that are expressed as ranged can assume any specific value or sub-range within the stated ranges in different embodiments of the invention, to the tenth of the unit of the lower limit of the range, unless the context clearly dictates otherwise.

The term “about” or “approximately” means within an acceptable error range for the particular value as determined by one of the ordinary skill in the art, which will depend in part on how the value is measured or determined, e.g., the limitations of the measurement system. For example, “about” can mean within 1 or more than 1 standard deviation, per the practice in the art. Alternatively, “about” can mean a range of up to 20%, up to 10%, up to 5% or up to 1% of a given value. Alternatively, the term can mean within an order of magnitude, for example within 5-fold, or within 2-fold, of a value. Where particular values are described in the application and claims, unless otherwise stated the term “about” meaning within an acceptable error range for the particular value should be assumed.

In addition, it is to be understood that any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the method of the invention can be excluded from any one or more claims, for any reason, whether or not related to the existence of prior art.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

I claim

1. A method of treating an injured joint in a mammal, comprising administering an injectable composition comprising a pharmaceutically acceptable salt of metformin at a concentration of 25 mM to 150 mM to the injured joint via intraarticular injection.

2. The method of claim 1, wherein the pharmaceutically acceptable salt of metformin is metformin hydrochloride; and
wherein the injectable composition comprises a concentration of about 50 mM to 150 mM metformin hydrochloride, inclusive.

3. The method of claim 1, comprising administering 2 to 3 mL of the injectable composition to the injured joint via the intraarticular route.

4. The method of claim 1, wherein the injectable composition is administered via intraarticular administration to the injured joint within 1 week or within 2 weeks of the injury.

5. The method of claim 1, comprising administering the injectable composition by intraarticular administration to the injured joint within 1 hour, 12 hours, or 24 hours from occurrence of the injury.

6. The method of claim 1, comprising administering the injectable composition via intraarticular injection to an injured wrist joint, an injured shoulder joint, an injured knee joint, an injured hip joint, an injured ankle joint, or an injured elbow joint in the mammal.

7. The method of claim 1, wherein the injectable composition further comprises a hydrogel and a pharmaceutically acceptable excipient.

8. The method of claim 7, wherein the hydrogel comprises hyaluronic acid or a salt thereof, wherein the salt is sodium hyaluronate or potassium hyaluronate.

9. The method of claim 1, further comprising systemically administering an amount of a systemic formulation of metformin to the mammal, wherein the systemic formulation of metformin comprises a pharmaceutically acceptable salt of metformin selected from the group consisting of metformin benzoate, metformin ethanesulfonate, metformin (2:1) fumarate, metformin glycinate, metformin glycolate, metformin hydrobromide, metformin hydrochloride, metformin maleate, metformin phosphate, metformin salicylate, metformin (2:1) succinate, and metformin sulfate.

10. The method of claim 9, wherein the systemic formulation of metformin further comprises alogliptin, canagliflozin, dapagliflozin, empagliflozin, ertugliflozin, glipizide, glyburide, linagliptin, pharmaceutically acceptable salts thereof, or a combination thereof.

11. The method of claim 9, wherein the amount of the pharmaceutically acceptable salt of metformin in the systemic formulation is up to about 100 mg to 2000 mg, 1000 mg, or 3000 mg.

12. The method of claim 9, wherein systemic administration comprises oral administration, intramuscular (IM) administration, intravenous (IV) administration, or a combination thereof.

13. The method of claim 1, wherein the injured joint is a wrist joint, a shoulder joint, a knee joint, a hip joint, an ankle joint, or an elbow joint, and
wherein the injured joint comprises a fracture, cartilage damage, acute ligament sprain, chronic ligamentous instability, tendonitis, or a combination thereof.

14. The method of claim 9, further comprising administering the injectable composition of metformin and/or the systemic formulation of metformin to a joint in a mammal prior to joint injury, wherein the joint is any one or more of a wrist joint, a shoulder joint, a knee joint, a hip joint, an ankle joint, and an elbow joint.

15. The method of claim 1, wherein at least one symptom of post-traumatic osteoarthritis is prevented or reduced in the injured joint following treatment with the pharmaceutically acceptable salt of metformin, wherein the at least one symptom is cartilage degeneration, joint pain, joint stiffness, inflammation, or a combination thereof.

16. The method of claim 15, wherein prevention or reduction of the at least one symptom of post-traumatic osteoarthritis is determined by the presence of biomarkers, gait analysis, imaging analysis, histological analysis, a self-reported scale, or a combination thereof.

17. The method of claim 1, wherein the mammal is human.

18. An injectable composition, consisting essentially of hyaluronic acid or a salt thereof, wherein the salt is sodium hyaluronate or potassium hyaluronate, and at least 50 mM of a pharmaceutically acceptable salt of metformin, wherein the pharmaceutically acceptable salt of metformin is metformin hydrochloride, and the composition is suitable for intraarticular injection.

19. The injectable composition of claim 18, comprising 50 mM to 150 mM metformin hydrochloride, inclusive.

20. The injectable composition of claim 18, wherein intraarticular injection of the composition prevents at least one symptom of post-traumatic osteoarthritis in a mammal with an injured joint, wherein the at least one symptom is cartilage degeneration, joint pain, joint stiffness inflammation, or a combination thereof.
